(19) United States
(12) Patent Application Publication
(10) Pub. No.: US 2008/0045534 A1
(43) Pub. Date: Feb. 21, 2008

(54) Derivatives of 1,3-diamino benzene as potassium channel modulators

(75) Inventors: Jean-Michel Vernier, Laguna Niguel, CA (US); Samedy Ouk, Costa Mesa, CA (US); Martha Alicia De La Rosa, Santa Ana, CA (US); David Abraham Paisner, Long Beach, CA (US)

Correspondence Address: SCULLY, SCOTT, MURPHY & PRESSER, P.C., 400 GARDEN CITY PLAZA, SUITE 300, GARDEN CITY, NY, 11530, US

(73) Assignee: Valeant Pharmaceuticals North America, Aliso Viejo, CA (US)

(21) Appl. No.: 11/893,724

(22) Filed: Aug. 16, 2007

Related U.S. Application Data
(60) Provisional application No. 60/838,808, filed on Aug. 18, 2006.
(60) Provisional application No. 60/839,642, filed on Aug. 22, 2006.

(51) Int. Cl. A61K 31/472; A61K 31/167; A61K 31/5375; A61P 25/00; C07C 233/43; C07D 217/04; C07D 295/14

(52) U.S. Cl. 514/237.8; 514/307; 514/613; 514/630; 544/166; 546/144; 564/189; 564/220

(57) ABSTRACT

This invention provides compounds of formula I

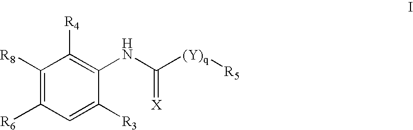

where X═O or S; Y is O or S; q=1 or 0; and other substituents are defined herein. The invention also provides pharmaceutical compositions comprising compounds of formula I. Such compounds are potassium channel modulators.

### CROSS REFERENCE TO RELATED APPLICATIONS

This application claims benefit of U.S. Provisional Application Nos. 60/838,808, filed Aug. 18, 2006, and 60/839,642, filed Aug. 22, 2006.

### FIELD OF THE INVENTION

This invention concerns novel compounds that modulate potassium channels. The compounds are useful for the treatment and prevention of diseases and disorders which are affected by modulation of potassium ion channels. One such condition is seizure disorders.

### BACKGROUND OF THE INVENTION

Retigabine (N-[2-amino-4-(4-fluorobenzylamino)phenyl]carbamic acid, ethyl ester] (U.S. Pat. No. 5,384,330) has been found to be an effective treatment of seizure disorders. Bialer, M. et al., Epilepsy Research 1999, 34, 1-41. Retigabine has also been found to be useful in treating pain, including neuropathic pain. Blackburn-Munro and Jensen, Eur. J. Pharmacol. 2003, 460, 109-116; Wickenden, A. D. et al., Expert Opin. Ther. Patents, 2004, 14(4).

“Benign familial neonatal convulsions,” an inherited form of epilepsy, has been associated with mutations in the KCNQ2/3 channels. Biervert, C. et al., Science 1998, 27, 403-06; Singh, N. A., et al., Nat. Genet. 1998, 18, 25-29; Charlier, C. et al., Nat. Genet. 1998, 18, 53-55; Rogawski, Trends in Neurosciences 2000, 23, 393-398. Subsequent investigations have established that one important site of action of retigabine is the KCNQ2/3 channel. Wickenden, A. D. et al., Mol. Pharmacol. 2000, 58,591-600; Main, M. J. et al., Mol. Pharmcol. 2000, 58, 253-62. Retigabine has been shown to increase the conductance of the channels at the resting membrane potential, with a possible mechanism involving binding of the activation gate of the KCNQ 2/3 channel. Wuttke, T. V., et al., Mol. Pharmacol. 2005, 67, 1009-1017. With increased sophistication of research in this area, retigabine has also been shown to increase neuronal M currents and to increase the channel open probability of KCNQ 2/3 channels. Delmas, P. and Brown, D .A. Nat. Revs Neurosci., vol. 6, 2005, 850-62; Tatulian, L. and Brown, D. A., J. Physiol., (2003) 549, 57-63.

The recognition of the site of action of retigabine has prompted a search for other potassium channel activators and modulators among compounds related to retigabine.

### BRIEF DESCRIPTION OF THE INVENTION

In one embodiment, this invention provides compound of formula I

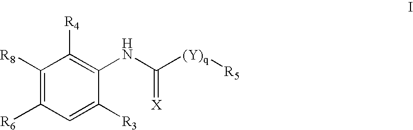

where R₈ is one of Groups A-C below

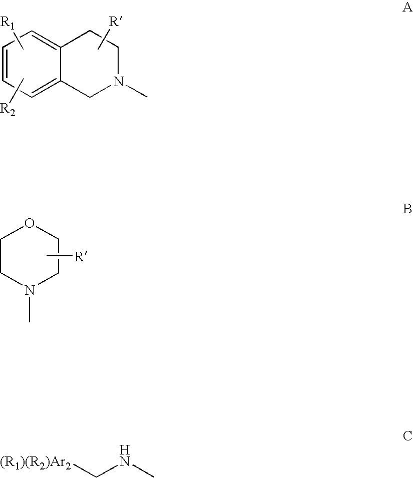

where R₁ and R₂, are, independently, H, CN, halogen, CH₂CN, OH, NO₂, CH₂F, CHF₂, CF₃, CF₂CF₃, C₁-C₆ alkyl, C(═O)C₁-C₆ alkyl, NH—C₁-C₆ alkyl, NHC(═O)C₁-C₆ alkyl, C(═O)N(CH₃)₂, C(═O)N(Et)₂, C(═O)NH—C₁-C₆alkyl, C(═O)OC₁-C₆alkyl, OC(═O)C₁-C₆ alkyl, OC₁-C₆ alkyl, SC₁-C₆ alkyl, C₃-C₆ cycloalkyl, (CH₂)ₘC₃-C₆ cycloalkyl, C₃-C₆ cycloalkenyl, (CH₂)ₘC₃-C₆ cycloalkenyl, C₂-C₆ alkenyl, C₂-C₆ alkynyl, phenyl, pyridyl, pyrrolyl, (CH₂)ₘimidazolyl, (CH₂)ₘpyrazyl, (CH₂)ₘoxazolyl, (CH₂)ₘisoxazolyl, (CH₂)ₘthiazolyl, (CH₂)ₘisothiazolyl, (CH₂)ₘphenyl, (CH₂)ₘpyrrolyl, (CH₂)ₘpyridyl, or (CH₂)ₘpyrimidyl, which cycloalkyl and said cycloalkenyl groups optionally contain one or two heteroatoms selected independently from O, N, and S, and which alkyl, cycloalkyl, cycloalkenyl, alkenyl, alkynyl, imidazolyl, pyrazyl, oxazolyl, isoxazolyl, thiazolyl, isothiazolyl, phenyl, pyrrolyl, pyridyl, or pyrimidyl groups are optionally substituted with one or two groups selected, independently, from OH, halogen, cyano, methyl, ethyl, or trifluoromethyl, where m is zero, 1, or 2; or R₁ and R₂, together with the ring carbon atoms to which they are attached, form a 5- or 6-member fused ring, which ring may be saturated, unsaturated, or aromatic, which optionally contains one or two heteroatoms selected independently from O, N, and S, and which is optionally substituted with halogen, CF₃, or C₁-C₃ alkyl; R′ is H, halogen, CF₃, or C₁-C₃ alkyl; R₃, R₄, and R₆ are, independently, H, CN, halogen, CF₃, OCF₃, OC₁-C₃ alkyl, or C₁-C₆ alkyl, all said C₁-C₃ alkyl groups and said C₁-C₆ alkyl groups optionally substituted with one or two groups selected, independently, from OH, halogen, C₁-C₃ alkyl, OC₁-C₃ alkyl, or trifluoromethyl; X═O or S; Y is O or S; q=1 or 0; R₅ is C₁-C₆alkyl, (CHR₇)_{w}C₃-C₆ cycloalkyl, (CHR₇)_{w}CH₂C₃-C₆ cycloalkyl, CH₂(CHR₇)_{w}C₃-C₆ cycloalkyl, CR₇═CH—C₃-C₆ cycloalkyl, CH═CR₇—C₃-C₆ cycloalkyl, (CHR₇)_{w}C₅-C₆ cycloalkenyl, CH₂(CHR₇)_{w}C₅-C₆ cycloalkenyl, C₂-C₆ alkenyl, C₂-C₆ alkynyl, Ar₁, (CHR₇)_{w}Ar₁, CH₂(CHR₇)_{w}Ar₁, or (CHR₇)_{w}CH₂Ar₁, where w=0-3, Ar₁ is a 5- to 10-member mono- or bicyclic aromatic group, optionally containing 1-4 ring heteroatoms selected independently from N, O, and S; R₇ is C₁-C₃ alkyl or hydrogen; Ar₂ is a 5- to 10-member mono- or bicyclic aromatic group, optionally containing 1-4 heteroatoms selected independently from N, O, and S, where all alkyl, cycloalkyl, alkenyl, cycloalkenyl, heterocycloalkyl, heterocycloalkenyl, alkynyl, aryl, and heteroaryl groups in R₁, R₂, R₃, R₄, R₅, R₆, R₇, Ar₁, and Ar₂ are optionally substituted with one or two substituents selected independently from C₁-C₃ alkyl, halogen, OH, OMe, CN, CH₂F, and trifluoromethyl; where, additionally, all cycloalkyl and heterocycloalkyl groups are optionally substituted with either an exocyclic carbon-carbon double bond or a carbonyl group; and where, additionally, the alkenyl and alkynyl groups are also optionally substituted with phenyl or C₃-C₆ cycloalkyl; or a pharmaceutically acceptable salt thereof. Such compounds are potassium channel modulators.

In another embodiment, the present invention provides a pharmaceutical composition comprising a compound of formula I and a pharmaceutically acceptable carrier, excipient, or diluent.

In a further embodiment, the present invention provides a composition comprising a pharmaceutically acceptable carrier, excipient, or diluent and at least one of the following: a pharmaceutically effective amount of a compound of formula I and a pharmaceutically acceptable salt or ester thereof.

In another embodiment, the present invention provides a method of preventing or treating a disease or disorder which is affected by modulation of voltage-gated potassium channels, comprising administering to a patient in need thereof a therapeutically effective amount of a composition comprising one or more of the following: a compound of formula I, a salt of a compound of formula I, and an ester of a compound of formula I.

In another embodiment, this invention provides a composition comprising a pharmaceutically acceptable carrier, excipient, or diluent and one or more of the following: a pharmaceutically effective amount of a compound of formula I; a pharmaceutically effective amount of a pharmaceutically acceptable salt thereof, a pharmaceutically effective amount of a pharmaceutically acceptable ester thereof.

In yet another embodiment, this invention provides a method of preventing or treating a disease or disorder which is affected by modulation of voltage-gated potassium channels, comprising administering to a patient in need thereof a therapeutically effective amount of a compound of formula I or a salt or ester thereof.

In another embodiment, this invention provides or contemplates a method of treating or preventing a disease or disorder which is affected by enhancement of neural M currents comprising administering to a patient in need thereof one or more of the following: i) a pharmaceutically effective amount of a compound of formula I; ii) a pharmaceutically acceptable salt thereof; iii) a pharmaceutically acceptable ester thereof; iv) and a pharmaceutically acceptable solvate thereof.

In yet another embodiment, this invention provides a method of preventing or treating a disease or disorder which is affected by activation of voltage-gated potassium channels, comprising administering to a patient in need thereof one or more of the following: a pharmaceutically effective amount of a compound of formula I; ii) a pharmaceutically acceptable salt thereof; iii) a pharmaceutically acceptable ester thereof; and iv) a pharmaceutically acceptable solvate thereof.

In yet another embodiment, this invention provides or contemplates a method of treating or preventing a seizure disorder in a human comprising administering to a patient afflicted or potentially afflicted with such disorder one or more of the following: a pharmaceutically effective amount of a compound of formula I; ii) a pharmaceutically acceptable salt thereof; iii) a pharmaceutically acceptable ester thereof; iv) and a pharmaceutically acceptable solvate thereof.

In another embodiment, this invention provides or contemplates a pharmaceutical formulation for oral administration comprising a therapeutically effective amount of a compound of formula I and either an appropriate tabletting agent or an appropriate syrup for pediatric use.

In another embodiment, this invention provides or contemplates a tablet for oral administration comprising a therapeutically effective amount of a compound of formula I and an appropriate tabletting agent.

In another appropriate embodiment, this invention provides or contemplates a syrup for pediatric use comprising a solution or dispersion or suspension of a compound of formula I and an appropriate syrup.

In another embodiment, this invention contemplates a pharmaceutical formulation for administration to animals, including companion animals (dogs and cats), and livestock comprising a therapeutically effective amount of a compound of formula I.

In another embodiment, this invention contemplates a method of preventing or treating in an animal a disease or disorder which is affected by activation of voltage-gated potassium channels comprising administering to an animal in need thereof one or more of the following: i) a pharmaceutically effective amount of a compound of formula I; ii) a pharmaceutically acceptable salt thereof; iii) a pharmaceutically acceptable ester thereof; iv) and a pharmaceutically acceptable solvate thereof.

This invention includes all tautomers, salts, and stereoisomeric forms of compounds of formula I. This invention also includes all compounds of this invention where one or more atoms are replaced by a radioactive isotope thereof.

This invention provides or contemplates compounds of formula I above where NH—C(═X)—(Y)_{q}—R₅ is each of the following: NHC(═O)R_{5,} NHC(═O)OR₅, NHC(═S)R₅, NHC(═S)SR₅, NHC(═S)OR₅, and NHC(═O)SR₅.

Thus, in one embodiment, this invention provides a compound of formula I, where NH—C(═X)—(Y)_{q}—R₅ is NHC(═O)R₅.

In another embodiment, this invention provides a compound of formula I, where NH—C(═X)—(Y)_{q}—R₅ is NHC(═S)R₅.

In another embodiment, this invention provides a compound of formula I, where NH—C(═X)—(Y)_{q}—R₅ is NHC(═S)SR₅.

In another embodiment, this invention provides a compound of formula I, where NH—C(═X)—(Y)_{q}—R₅ is each NHC(═O)OR₅.

In another embodiment, this invention provides a compound of formula I, where NH—C(═X)—(Y)_{q}—R₅ is NHC(═S)OR₅.

In another embodiment, this invention provides a compound of formula I, where NH—C(═X)—(Y)_{q}—R₅ is NHC(═O)SR₅.

In one subgeneric embodiment, this invention provides a compound of formula I, where R₈ is Group A and NH—C(═X)—(Y)_{q}—R₅ is NHC(═O)R₅ or NHC(═S)R₅.

In another subgeneric embodiment, this invention provides a compound of formula I, where R₈ is Group A and NH—C(═X)—(Y)_{q}—R₅ is NHC(═O)SR₅ or NHC(═S)OR₅.

In another subgeneric embodiment, this invention provides a compound of formula I, where R₈ is Group A and NH—C(═X)—(Y)_{q}—R₅ is NHC(═O)OR₅ or NHC(═S)SR₅.

In another subgeneric embodiment, this invention provides a compound of formula I, where R₈ is Group B and NH—C(═X)—(Y)_{q}—R₅ is NHC(═O)R₅ or NHC(═S)R₅.

In another subgeneric embodiment, this invention provides a compound of formula I, where R₈ is Group B and NH—C(═X)—(Y)_{q}—R₅ is NHC(═O)SR₅ or NHC(═S)OR₅.

In another subgeneric embodiment, this invention provides a compound of formula I, where R₈ is Group B and NH—C(═X)—(Y)_{q}—R₅ is NHC(═O)OR₅ or NHC(═S)SR₅.

In another subgeneric embodiment, this invention provides a compound of formula I, where R₈ is Group C and NH—C(═X)—(Y)_{q}—R₅ is NHC(═O)R₅ or NHC(═S)R₅.

In another subgeneric embodiment, this invention provides a compound of formula I, where R₈ is Group C and NH—C(═X)—(Y)_{q}—R₅ is NHC(═O)SR₅ or NHC(═S)OR₅.

In another subgeneric embodiment, this invention provides a compound of formula I, where R₈ is Group C and NH—C(═X)—(Y)_{q}—R₅ is NHC(═O)OR₅ or NHC(═S)SR₅.

In another subgeneric embodiment, this invention provides a compound of formula IA below.

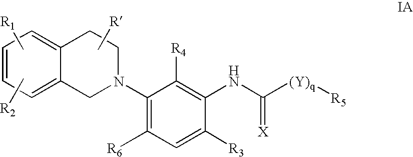

In another subgeneric embodiment, this invention provides a compound of formula IB below.

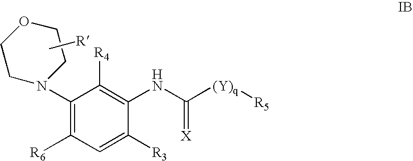

In another subgeneric embodiment, this invention provides a compound of formula IC below.

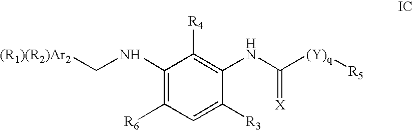

In a more specific subgeneric embodiment, this invention provides or contemplates a compound of formula IA, where X is O, q=1, Y is O, and R₅ is C₁-C₆ alkyl, (CHR₇)_{w}C₃-C₆ cycloalkyl, (CHR₇)_{w}CH₂C₃-C₆ cycloalkyl, or CH₂(CHR₇)_{w}C₃-C₆ cycloalkyl.

In another more specific subgeneric embodiment, this invention provides or contemplates a compound of formula IA, where X is O, q=1, Y is O, and R₅ is CR₇═CH—C₃-C₆ cycloalkyl, CH═CR₇—C₃-C₆ cycloalkyl, (CHR₇)_{w}C₅-C₆ cycloalkenyl, CH₂(CHR₇)_{w}C₅-C₆ cycloalkenyl, C₂-C₆ alkenyl, or C₂-C₆ alkynyl.

In another more specific subgeneric embodiment, this invention provides or contemplates a compound of formula IA, where X is O, q=1, Y is O, and R₅ is Ar₁, (CHR₇)_{w}Ar₁, CH₂(CHR₇)_{w}Ar₁, or (CHR₇)_{w}CH₂Ar₁.

In another more specific subgeneric embodiment, this invention provides or contemplates a compound of formula IA, where X is O, q=1, Y is S, and R₅ is C₁-C₆ alkyl, (CHR₇)_{w}C₃-C₆ cycloalkyl, (CHR₇)_{w}CH₂C₃-C₆ cycloalkyl, or CH₂(CHR₇)_{w}C₃-C₆ cycloalkyl.

In another more specific subgeneric embodiment, this invention provides or contemplates a compound of formula IA, where X is O, q=1, Y is S, and R₅ is CR₇═CH—C₃-C₆ cycloalkyl, CH═CR₇—C₃-C₆ cycloalkyl, (CHR₇)_{w}C₅-C₆ cycloalkenyl, CH₂(CHR₇)_{w}C₅-C₆ cycloalkenyl, C₂-C₆ alkenyl, or C₂-C₆ alkynyl.

In another more specific subgeneric embodiment, this invention provides or contemplates a compound of formula IA, where X is O, q=1, Y is S, and R₅ is Ar₁, (CHR₇)_{w}Ar₁, CH₂(CHR₇)_{w}Ar₁, or (CHR₇)_{w}CH₂Ar₁.

In another more specific subgeneric embodiment, this invention provides or contemplates a compound of formula IA, where X is O, q=zero, and R₅ is C₁-C₆ alkyl, (CHR₇)_{w}C₃-C₆ cycloalkyl, (CHR₇)_{w}CH₂C₃-C₆ cycloalkyl, or CH₂(CHR₇)_{w}C₃-C₆ cycloalkyl.

In another more specific subgeneric embodiment, this invention provides or contemplates a compound of formula IA, where X is O, q=zero, and R₅ is CR₇═CH—C₃-C₆ cycloalkyl, CH═CR₇—C₃-C₆ cycloalkyl, (CHR₇)_{w}C₅-C₆ cycloalkenyl, CH₂(CHR₇)_{w}C₅-C₆ cycloalkenyl, C₂-C₆ alkenyl, or C₂-C₆ alkynyl.

In another more specific subgeneric embodiment, this invention provides or contemplates a compound of formula IA, where X is O, q=zero, and R₅ is Ar₁, (CHR₇)_{w}Ar₁, CH₂(CHR₇)_{w}Ar₁, or (CHR₇)_{w}CH₂Ar₁.

In another more specific subgeneric embodiment, this invention provides or contemplates a compound of formula IA, where X is S, q=1, Y is O, and R₅ is C₁-C₆ alkyl, (CHR₇)_{w}C₃-C₆ cycloalkyl, (CHR₇)_{w}CH₂C₃-C₆ cycloalkyl, or CH₂(CHR₇)_{w}C₃-C₆ cycloalkyl.

In another more specific subgeneric embodiment, this invention provides or contemplates a compound of formula IA, where X is S, q=1, Y is O, and R₅ is CR₇═CH—C₃-C₆ cycloalkyl, CH═CR₇—C₃-C₆ cycloalkyl, (CHR₇)_{w}C₅-C₆ cycloalkenyl, CH₂(CHR₇)_{w}C₅-C₆ cycloalkenyl, C₂-C₆ alkenyl, or C₂-C₆ alkynyl.

In another more specific subgeneric embodiment, this invention provides or contemplates a compound of formula IA, where X is S, q=1, Y is O, and R₅ is Ar₁, (CHR₇)_{w}Ar₁, CH₂(CHR₇)_{w}Ar₁, or (CHR₇)_{w}CH₂Ar₁.

In another more specific subgeneric embodiment, this invention provides or contemplates a compound of formula IA, where X is S, q=zero, and R₅ is C₁-C₆ alkyl, (CHR₇)_{w}C₃-C₆ cycloalkyl, (CHR₇)_{w}CH₂C₃-C₆ cycloalkyl, or CH₂(CHR₇)_{w}C₃-C₆ cycloalkyl.

In another more specific subgeneric embodiment, this invention provides or contemplates a compound of formula IA, where X is S, q=zero, and R₅ is CR₇═CH—C₃-C₆ cycloalkyl, CH═CR₇—C₃-C₆ cycloalkyl, (CHR₇)_{w}C₅-C₆ cycloalkenyl, CH₂(CHR₇)_{w}C₅-C₆ cycloalkenyl, C₂-C₆ alkenyl, or C₂-C₆ alkynyl.

In another more specific subgeneric embodiment, this invention provides or contemplates a compound of formula IA, where X is S, q=zero, and R₅ is Ar₁, (CHR₇)_{w}Ar₁, CH₂(CHR₇)_{w}Ar₁, or (CHR₇)_{w}CH₂Ar₁.

In another more specific subgeneric embodiment, this invention provides or contemplates a compound of formula IA, where X is S, q=1, Y is S, and R₅ is C₁-C₆ alkyl, (CHR₇)_{w}C₃-C₆ cycloalkyl, (CHR₇)_{w}CH₂C₃-C₆ cycloalkyl, or CH₂(CHR₇)_{w}C₃-C₆ cycloalkyl.

In another more specific subgeneric embodiment, this invention provides or contemplates a compound of formula IA, where X is S, q=1, Y is S, and R₅ is CR₇═CH—C₃-C₆ cycloalkyl, CH═CR₇—C₃-C₆ cycloalkyl, (CHR₇)_{w}C₅-C₆ cycloalkenyl, CH₂(CHR₇)_{w}C₅-C₆ cycloalkenyl, C₂-C₆ alkenyl, or C₂-C₆ alkynyl.

In another more specific subgeneric embodiment, this invention provides or contemplates a compound of formula IA, where X is S, q=1, Y is S, and R₅ is Ar₁, (CHR₇)_{w}Ar₁, CH₂(CHR₇)_{w}Ar₁, or (CHR₇)_{w}CH₂Ar₁.

In another more specific subgeneric embodiment, this invention provides or contemplates a compound of formula IB, where X is O, q=1, Y is O, and R₅ is C₁-C₆ alkyl, (CHR₇)_{w}C₃-C₆ cycloalkyl, (CHR₇)_{w}CH₂C₃-C₆ cycloalkyl, or CH₂(CHR₇)_{w}C₃-C₆ cycloalkyl.

In another more specific subgeneric embodiment, this invention provides or contemplates a compound of formula IB, where X is O, q=1, Y is O, and R₅ is CR₇═CH—C₃-C₆ cycloalkyl, CH═CR₇—C₃-C₆ cycloalkyl, (CHR₇)_{w}C₅-C₆ cycloalkenyl, CH₂(CHR₇)_{w}C₅-C₆ cycloalkenyl, C₂-C₆ alkenyl, or C₂-C₆ alkynyl.

In another more specific subgeneric embodiment, this invention provides or contemplates a compound of formula IB, where X is O, q=1, Y is O, and R₅ is Ar₁, (CHR₇)_{w}Ar₁, CH₂(CHR₇)_{w}Ar₁, or (CHR₇)_{w}CH₂Ar₁.

In another more specific subgeneric embodiment, this invention provides or contemplates a compound of formula IB, where X is O, q=1, Y is S, and R₅ is C₁-C₆ alkyl, (CHR₇)_{w}C₃-C₆ cycloalkyl, (CHR₇)_{w}CH₂C₃-C₆ cycloalkyl, or CH₂(CHR₇)_{w}C₃-C₆ cycloalkyl.

In another more specific subgeneric embodiment, this invention provides or contemplates a compound of formula IB, where X is O, q=1, Y is S, and R₅ is CR₇═CH—C₃-C₆ cycloalkyl, CH═CR₇—C₃-C₆ cycloalkyl, (CHR₇)_{w}C₅-C₆ cycloalkenyl, CH₂(CHR₇)_{w}C₅-C₆ cycloalkenyl, C₂-C₆ alkenyl, or C₂-C₆ alkynyl.

In another more specific subgeneric embodiment, this invention provides or contemplates a compound of formula IB, where X is O, q=1, Y is S, and R₅ is Ar₁, (CHR₇)_{w}Ar₁, CH₂(CHR₇)_{w}Ar₁, or (CHR₇)_{w}CH₂Ar₁.

In another more specific subgeneric embodiment, this invention provides or contemplates a compound of formula IB, where X is O, q=zero, and R₅ is C₁-C₆ alkyl, (CHR₇)_{w}C₃-C₆ cycloalkyl, (CHR₇)_{w}CH₂C₃-C₆ cycloalkyl, or CH₂(CHR₇)_{w}C₃-C₆ cycloalkyl.

In another more specific subgeneric embodiment, this invention provides or contemplates a compound of formula IB, where X is O, q=zero, and R₅ is CR₇═CH—C₃-C₆ cycloalkyl, CH═CR₇—C₃-C₆ cycloalkyl, (CHR₇)_{w}C₅-C₆ cycloalkenyl, CH₂(CHR₇)_{w}C₅-C₆ cycloalkenyl, C₂-C₆ alkenyl, or C₂-C₆ alkynyl.

In another more specific subgeneric embodiment, this invention provides or contemplates a compound of formula IB, where X is O, q=zero, and R₅ is Ar₁, (CHR₇)_{w}Ar₁, CH₂(CHR₇)_{w}Ar₁, or (CHR₇)_{w}CH₂Ar₁.

In another more specific subgeneric embodiment, this invention provides or contemplates a compound of formula IB, where X is S, q=1, Y is O, and R₅ is C₁-C₆ alkyl, (CHR₇)_{w}C₃-C₆ cycloalkyl, (CHR₇)_{w}CH₂C₃-C₆ cycloalkyl, or CH₂(CHR₇)_{w}C₃-C₆ cycloalkyl.

In another more specific subgeneric embodiment, this invention provides or contemplates a compound of formula IB, where X is S, q=1, Y is O, and R₅ is CR₇═CH—C₃-C₆ cycloalkyl, CH═CR₇—C₃-C₆ cycloalkyl, (CHR₇)_{w}C₅-C₆ cycloalkenyl, CH₂(CHR₇)_{w}C₅-C₆ cycloalkenyl, C₂-C₆ alkenyl, or C₂-C₆ alkynyl.

In another more specific subgeneric embodiment, this invention provides or contemplates a compound of formula IB, where X is S, q=1, Y is O, and R₅ is Ar₁, (CHR₇)_{w}Ar₁, CH₂(CHR₇)_{w}Ar₁, or (CHR₇)_{w}CH₂Ar₁.

In another more specific subgeneric embodiment, this invention provides or contemplates a compound of formula IB, where X is S, q=zero, and R₅ is C₁-C₆ alkyl, (CHR₇)_{w}C₃-C₆ cycloalkyl, (CHR₇)_{w}CH₂C₃-C₆ cycloalkyl, or CH₂(CHR₇)_{w}C₃-C₆ cycloalkyl.

In another more specific subgeneric embodiment, this invention provides or contemplates a compound of formula IB, where X is S, q=zero, and R₅ is CR₇═CH—C₃-C₆ cycloalkyl, CH═CR₇—C₃-C₆ cycloalkyl, (CHR₇)_{w}C₅-C₆ cycloalkenyl, CH₂(CHR₇)_{w}C₅-C₆ cycloalkenyl, C₂-C₆ alkenyl, or C₂-C₆ alkynyl.

In another more specific subgeneric embodiment, this invention provides or contemplates a compound of formula IB, where X is S, q=zero, and R₅ is Ar₁, (CHR₇)_{w}Ar₁, CH₂(CHR₇)_{w}Ar₁, or (CHR₇)_{w}CH₂Ar₁.

In another more specific subgeneric embodiment, this invention provides or contemplates a compound of formula IB, where X is S, q=1, Y is S, and R₅ is C₁-C₆ alkyl, (CHR₇)_{w}C₃-C₆ cycloalkyl, (CHR₇)_{w}CH₂C₃-C₆ cycloalkyl, or CH₂(CHR₇)_{w}C₃-C₆ cycloalkyl.

In another more specific subgeneric embodiment, this invention provides or contemplates a compound of formula IB, where X is S, q=1, Y is S, and R₅ is CR₇═CH—C₃-C₆ cycloalkyl, CH═CR₇—C₃-C₆ cycloalkyl, (CHR₇)_{w}C₅-C₆ cycloalkenyl, CH₂(CHR₇)_{w}C₅-C₆ cycloalkenyl, C₂-C₆ alkenyl, or C₂-C₆ alkynyl.

In another more specific subgeneric embodiment, this invention provides or contemplates a compound of formula IB, where X is S, q=1, Y is S, and R₅ is Ar₁, (CHR₇)_{w}Ar₁, CH₂(CHR₇)_{w}Ar₁, or (CHR₇)_{w}CH₂Ar₁.

In another more specific subgeneric embodiment, this invention provides or contemplates a compound of formula IC, where X is O, q=1, Y is O, and R₅ is C₁-C₆ alkyl, (CHR₇)_{w}C₃-C₆ cycloalkyl, (CHR₇)_{w}CH₂C₃-C₆ cycloalkyl, or CH₂(CHR₇)_{w}C₃-C₆ cycloalkyl.

In another more specific subgeneric embodiment, this invention provides or contemplates a compound of formula IC, where X is O, q=1, Y is O, and R₅ is CR₇═CH—C₃-C₆ cycloalkyl, CH═CR₇—C₃-C₆ cycloalkyl, (CHR₇)_{w}C₅-C₆ cycloalkenyl, CH₂(CHR₇)_{w}C₅-C₆ cycloalkenyl, C₂-C₆ alkenyl, or C₂-C₆ alkynyl.

In another more specific subgeneric embodiment, this invention provides or contemplates a compound of formula IC, where X is O, q=1, Y is O, and R₅ is Ar₁, (CHR₇)_{w}Ar₁, CH₂(CHR₇)_{w}Ar₁, or (CHR₇)_{w}CH₂Ar₁.

In another more specific subgeneric embodiment, this invention provides or contemplates a compound of formula IC, where X is O, q=1, Y is S, and R₅ is C₁-C₆ alkyl, (CHR₇)_{w}C₃-C₆ cycloalkyl, (CHR₇)_{w}CH₂C₃-C₆ cycloalkyl, or CH₂(CHR₇)_{w}C₃-C₆ cycloalkyl.

In another more specific subgeneric embodiment, this invention provides or contemplates a compound of formula IC, where X is O, q=1, Y is S, and R₅ is CR₇═CH—C₃-C₆ cycloalkyl, CH═CR₇—C₃-C₆ cycloalkyl, (CHR₇)_{w}C₅-C₆ cycloalkenyl, CH₂(CHR₇)_{w}C₅-C₆ cycloalkenyl, C₂-C₆ alkenyl, or C₂-C₆ alkynyl.

In another more specific subgeneric embodiment, this invention provides or contemplates a compound of formula IC, where X is O, q=1, Y is S, and R₅ is Ar₁, (CHR₇)_{w}Ar₁, CH₂(CHR₇)_{w}Ar₁, or (CHR₇)_{w}CH₂Ar₁.

In another more specific subgeneric embodiment, this invention provides or contemplates a compound of formula IC, where X is O, q=zero, and R₅ is C₁-C₆ alkyl, (CHR₇)_{w}C₃-C₆ cycloalkyl, (CHR₇)_{w}CH₂C₃-C₆ cycloalkyl, or CH₂(CHR₇)_{w}C₃-C₆ cycloalkyl.

In another more specific subgeneric embodiment, this invention provides or contemplates a compound of formula IC, where X is O, q=zero, and R₅ is CR₇═CH—C₃-C₆ cycloalkyl, CH═CR₇—C₃-C₆ cycloalkyl, (CHR₇)_{w}C₅-C₆ cycloalkenyl, CH₂(CHR₇)_{w}C₅-C₆ cycloalkenyl, C₂-C₆ alkenyl, or C₂-C₆ alkynyl.

In another more specific subgeneric embodiment, this invention provides or contemplates a compound of formula IC, where X is O, q=zero, and R₅ is Ar₁, (CHR₇)_{w}Ar₁, CH₂(CHR₇)_{w}Ar₁, or (CHR₇)_{w}CH₂Ar₁.

In another more specific subgeneric embodiment, this invention provides or contemplates a compound of formula IC, where X is S, q=1, Y is O, and R₅ is C₁-C₆ alkyl, (CHR₇)_{w}C₃-C₆ cycloalkyl, (CHR₇)_{w}CH₂C₃-C₆ cycloalkyl, or CH₂(CHR₇)_{w}C₃-C₆ cycloalkyl.

In another more specific subgeneric embodiment, this invention provides or contemplates a compound of formula IC, where X is S, q=1, Y is O, and R₅ is CR₇═CH—C₃-C₆ cycloalkyl, CH═CR₇—C₃-C₆ cycloalkyl, (CHR₇)_{w}C₅-C₆ cycloalkenyl, CH₂(CHR₇)_{w}C₅-C₆ cycloalkenyl, C₂-C₆ alkenyl, or C₂-C₆ alkynyl.

In another more specific subgeneric embodiment, this invention provides or contemplates a compound of formula IC, where X is S, q=1, Y is O, and R₅ is Ar₁, (CHR₇)_{w}Ar₁, CH₂(CHR₇)_{w}Ar₁, or (CHR₇)_{w}CH₂Ar₁.

In another more specific subgeneric embodiment, this invention provides or contemplates a compound of formula IC, where X is S, q=zero, and R₅ is C₁-C₆ alkyl, (CHR₇)_{w}C₃-C₆ cycloalkyl, (CHR₇)_{w}CH₂C₃-C₆ cycloalkyl, or CH₂(CHR₇)_{w}C₃-C₆ cycloalkyl.

In another more specific subgeneric embodiment, this invention provides or contemplates a compound of formula IC, where X is S, q=zero, and R₅ is CR₇═CH—C₃-C₆ cycloalkyl, CH═CR₇—C₃-C₆ cycloalkyl, (CHR₇)_{w}C₅-C₆ cycloalkenyl, CH₂(CHR₇)_{w}C₅-C₆ cycloalkenyl, C₂-C₆ alkenyl, or C₂-C₆ alkynyl.

In another more specific subgeneric embodiment, this invention provides or contemplates a compound of formula IC, where X is S, q=zero, and R₅ is Ar₁, (CHR₇)_{w}Ar₁, CH₂(CHR₇)_{w}Ar₁, or (CHR₇)_{w}CH₂Ar₁.

In another more specific subgeneric embodiment, this invention provides or contemplates a compound of formula IC, where X is S, q=1, Y is S, and R₅ is C₁-C₆ alkyl, (CHR₇)_{w}C₃-C₆ cycloalkyl, (CHR₇)_{w}CH₂C₃-C₆ cycloalkyl, or CH₂(CHR₇)_{w}C₃-C₆ cycloalkyl.

In another more specific subgeneric embodiment, this invention provides or contemplates a compound of formula IC, where X is S, q=1, Y is S, and R₅ is CR₇═CH—C₃-C₆ cycloalkyl, CH═CR₇—C₃-C₆ cycloalkyl, (CHR₇)_{w}C₅-C₆ cycloalkenyl, CH₂(CHR₇)_{w}C₅-C₆ cycloalkenyl, C₂-C₆ alkenyl, or C₂-C₆ alkynyl.

In another more specific subgeneric embodiment, this invention provides or contemplates a compound of formula IC, where X is S, q=1, Y is S, and R₅ is Ar₁, (CHR₇)_{w}Ar₁, CH₂(CHR₇)_{w}Ar₁, or (CHR₇)_{w}CH₂Ar₁.

In another more specific subgeneric embodiment, this invention provides or contemplates a compound of formula IA, IB, or IC, where X is O, q=1, Y is O, and R₅ is C₁-C₆ alkyl or (CHR₇)_{w}C₃-C₆ cycloalkyl.

In another more specific subgeneric embodiment, this invention provides or contemplates a compound of formula IA, IB, or IC, where X is S, q=1, Y is S, and R₅ is C₁-C₆ alkyl or (CHR₇)_{w}C₃-C₆ cycloalkyl.

In another more specific subgeneric embodiment, this invention provides or contemplates a compound of formula IA, IB, or IC, where X is S, q=1, Y is O, and R₅ is C₁-C₆ alkyl or (CHR₇)_{w}C₃-C₆ cycloalkyl.

In another more specific subgeneric embodiment, this invention provides or contemplates a compound of formula IA, IB, or IC, where X is O, q=1, Y is S, and R₅ is C₁-C₆ alkyl or (CHR₇)_{w}C₃-C₆ cycloalkyl.

In another more specific subgeneric embodiment, this invention provides or contemplates a compound of formula IA, IB, or IC, where X is O, q=zero, and R₅ is C₁-C₆ alkyl or (CHR₇)_{w}C₃-C₆ cycloalkyl.

In a still more specific subgeneric embodiment, this invention provides or contemplates a compound of formula IA, IB, or IC, where X is O, q=zero, and R₅ is C₃-C₆ alkyl, CH₂CH₂— cyclopentyl or one of the groups below:

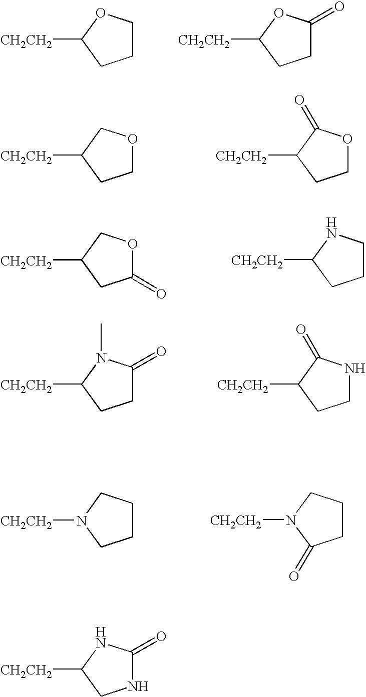

In another more specific subgeneric embodiment, this invention provides or contemplates a compound of formula IA, IB, or IC, where X is S, q=zero, and R₅ is C₁-C₆ alkyl or (CHR₇)_{w}C₃-C₆ cycloalkyl.

In another subgeneric embodiment, this invention provides or contemplates a compound of formula IA, IB, or IC, where R₁ is H, CN, halogen, CH₂CN, OH, NO₂, CH₂F, CHF₂, CF₃, CF₂CF₃, C₁-C₆ alkyl, or C(═O)C₁-C₆ alkyl.

In another subgeneric embodiment, this invention provides or contemplates a compound of formula IA, IB, or IC, where R₁ is C(═O)C₁-C₆alkyl, NHC(═O)C₁-C₆ alkyl, C(═O)N(CH₃)₂, C(═O)N(Et)₂, C(═O)NH—C₁-C₆ alkyl, C(═O)OC₁-C₆ alkyl, or OC(═O)C₁-C₆ alkyl.

In another subgeneric embodiment, this invention provides or contemplates a compound of formula IA, IB, or IC, where R₁ is OC₁-C₆ alkyl, SC₁-C₆ alkyl, C₃-C₆ cycloalkyl, (CH₂)ₘC₃-C₆ cycloalkyl, C₃-C₆ cycloalkenyl, (CH₂)ₘC₃-C₆ cycloalkenyl, C₂-C₆ alkenyl, or C₂-C₆ alkynyl.

In another subgeneric embodiment, this invention provides or contemplates a compound of formula IA, IB, or IC, where R₁ is phenyl, pyridyl, pyrrolyl, (CH₂)ₘpyrazyl, (CH₂)ₘimidazolyl, (CH₂)ₘoxazolyl, (CH₂)ₘisoxazolyl, (CH₂)ₘthiazolyl, (CH₂)ₘpyridyl, (CH₂)ₘisothiazolyl, (CH₂)ₘphenyl, (CH₂)ₘpyrrolyl, or (CH₂)ₘpyrimidyl.

In another more specific subgeneric embodiment, this invention provides or contemplates a compound of formula IA, IB, or IC, where R₁ is C(═O)C₁-C₆ alkyl, NHC(═O)C₁-C₆ alkyl, C(═O)N(CH₃)₂, C(═O)N(Et)₂, C(═O)NH—C₁-C₆ alkyl, C(═O)OC₁-C₆ alkyl, or OC(═O)C₁-C₆ alkyl, and R₅ is C₅-C₆ alkyl or CH₂—C₃-C₆ cycloalkyl.

In another more specific subgeneric embodiment, this invention provides or contemplates a compound of formula IA, IB, or IC, where R₁ is OC₁-C₆ alkyl, SC₁-C₆ alkyl, C₃-C₆ cycloalkyl, (CH₂)ₘC₃-C₆ cycloalkyl, C₃-C₆ cycloalkenyl, (CH₂)ₘC₃-C₆ cycloalkenyl, C₂-C₆ alkenyl, or C₂-C₆ alkynyl, and R₅ is C₅-C₆ alkyl or CH₂—C₃-C₆ cycloalkyl.

In another more specific subgeneric embodiment, this invention provides or contemplates a compound of formula IA, IB, or IC, where R₁ is phenyl, pyridyl, pyrrolyl, (CH₂)ₘimidazolyl, (CH₂)ₘpyrazyl, (CH₂)ₘoxazolyl, (CH₂)ₘisoxazolyl, (CH₂)ₘthiazolyl, (CH₂)ₘisothiazolyl, (CH₂)ₘphenyl, (CH₂)ₘpyrrolyl, (CH₂)ₘpyridyl, or (CH₂)ₘpyrimidyl, and R₅ is C₅-C₆ alkyl or CH₂—C₃-C₆ cycloalkyl.

In a more specific subgeneric embodiment, this invention provides or contemplates a compound of formula IA, where X is O, q=1, Y is O, and R₅ is Ar₁ or CH₂—Ar₁, where Ar₁ is unsubstituted phenyl, monosubstituted phenyl, unsubstituted pyridyl, or unsubstituted pyrrolyl.

In another more specific subgeneric embodiment, this invention provides or contemplates a compound of formula IA, where X is O, q=zero, and R₅ is Ar₁ or CH₂—Ar₁, where Ar₁ is unsubstituted phenyl, monosubstituted phenyl, unsubstituted pyridyl, or unsubstituted pyrrolyl.

In another subgeneric embodiment, this invention provides or contemplates a compound of formula IA or IC, where R₁ and R₂ form a fused phenyl group, X is O, q=1, Y is O, and R₅ is C₁-C₆ alkyl or (CHR₇)_{w}C₃-C₆ cycloalkyl.

In another subgeneric embodiment, this invention provides or contemplates a compound of formula IA or IC, where R₁ and R₂ form a fused pyridyl group, X is O, q=1, Y is O, and R₅ is C₁-C₆ alkyl or (CHR₇)_{w}C₃-C₆ cycloalkyl.

In another subgeneric embodiment, this invention provides or contemplates a compound of formula IA, IB, or IC, where R₁ is halogen, C₁-C₆ alkyl, mono-halo C₁-C₆ alkyl, CN, di-halo C₁-C₆ alkyl, CF₃, CN, or O—C₁-C₆ alkyl, and R₅ is C₅-C₆ alkyl or CH₂—C₃-C₆ cycloalkyl.

In another more specific subgeneric embodiment, this invention provides or contemplates a compound of formula IA, IB, or IC, where R₁ is halogen, cyano, CF₃, or methoxy, R₂ is H or methyl, R′ is H, halogen, or methyl, and R₅ is C₅-C₆ alkyl or CH₂—C₃-C₆ cycloalkyl.

In another more specific subgeneric embodiment, this invention provides or contemplates a compound of formula IA, IB, or IC, where R₁ is halogen; R₂ is H or methyl, R′ is H, halogen, or methyl; and R₅ is C₅-C₆ alkyl or CH₂—C₅-C₆ cycloalkyl.

In another more specific embodiment, this invention provides or contemplates a compound of formula I, where R₅ is CH₂Ar₁ or CH₂CH₂—Ar₁, where Ar₁ is o-, m-, or p-xylyl or o-, m-, or p-anisyl.

In another more specific embodiment, this invention provides or contemplates a compound of formula I, where R₅ is CH₂Ar₁ or CH₂CH₂—Ar₁, where Ar₁ is m- or p-cyanophenyl or m- or p-cyanomethyl phenyl

In another more specific embodiment, this invention provides a compound of formula I, where R₅ is CH₂Ar₁ or CH₂CH₂—Ar₁, where Ar₁ is 3,5-dichlorophenyl or 3,5-difluorophenyl.

In a more specific embodiment, this invention provides a compound of formula IA where X is O, R₁, R₂, R′, and R₃ are H, and q=zero.

In another embodiment, this invention provides a compound of formula IB where X is O, R₁, R₂, R′, and R₃ are H, and q=zero.

In another embodiment, this invention provides a compound of formula IC where X is O, R₁, R₂, R′, and R₃ are H, and q=zero.

In a further embodiment, the present invention provides a composition comprising a pharmaceutically acceptable carrier, excipient or diluent and at least one of the following: a pharmaceutically effective amount of a compound of formula I and a pharmaceutically acceptable salt or ester thereof.

In another embodiment, the present invention provides a method of preventing or treating a disease or disorder which is affected by modulation of voltage-gated potassium channels, comprising administering to a patient in need thereof a therapeutically effective amount of a composition comprising one or more of the following: a compound of formula I, a salt of a compound of formula I, and an ester of a compound of formula I.

In another embodiment, this invention contemplates a method of diagnosing a disease or disorder which is affected by modulation of potassium ion channels comprising administering to a patient suspected of having such disease or disorder a probe comprising an isotopically labeled compound of formula I and/or a salt or ester thereof.

### DETAILED DESCRIPTION OF THE INVENTION

The compositions of the present invention may be formulated in a conventional manner using one or more pharmaceutically acceptable carriers.

As used herein, the term “pharmaceutically acceptable carrier” comprises such excipients, binders, lubricants, tabletting agents, and disintegrants as are typically used in the art of formulation of pharmaceuticals. Examples of such agents include—but are not limited to—microcrystalline cellulose, lactose, starch, and dicalcium phosphate, and Providone. However, in view of the incompatibility of primary amines with lactose, this invention does not contemplate compositions in which active ingredients with primary amine groups are combined with lactose. Additionally, disintegrants such as sodium starch glycolate; lubricants such as stearic acid and SiO₂; and solubility enhancers such as cyclodextrins, among many other examples for each group, are contemplated. Such materials and the methods of using them are well known in the pharmaceutical art. Additional examples are provided in Kibbe, Handbook of Pharmaceutical Excipients, London, Pharmaceutical Press, 2000.

As used herein the term “potassium channel modulator” refers to a compound capable of causing an increase in potassium channel currents. It also refers to a compound capable of increasing the KCNQ2/3 channel open probability. For preliminary testing of compounds for potassium channel modulating ability, the inventors have employed the rubidium ion efflux test described below.

As contemplated by this invention, compounds of formula I are designed for oral dosing of up to approximately 2000 mg per day. Similarly, solutions and suspensions suitable for oral pediatric administration, comprising, in addition to compounds of formula I, a syrup such as sorbitol or propylene glycol, among many other examples, are also contemplated. Additionally, both chewable and non-chewable tablets comprising compounds of formula I, along with pharmaceutically acceptable tabletting agents and other pharmaceutically acceptable carriers and excipients, are also contemplated.

The invention also contemplates pharmaceutical formulations for oral or intravenous administration to animals, comprising a therapeutically effective amount of a compound of formula I and an acceptable carrier for use in veterinary medicine. Any animal that is susceptible to disorders that are amenable to treatment with potassium channel modulators is included within the scope of this invention.

### Preparation of Compounds

Section I. The preparation of compounds of formula V is outlined in Scheme 1.

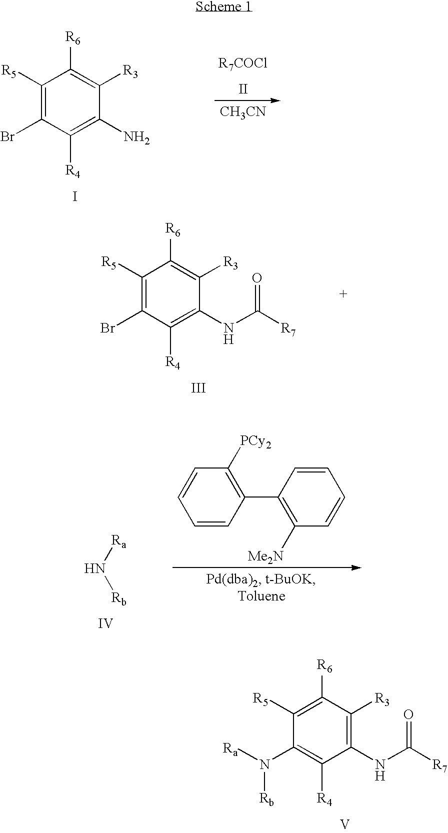

Section II. The preparation of compounds of formula VIII is outlined in Scheme 2.

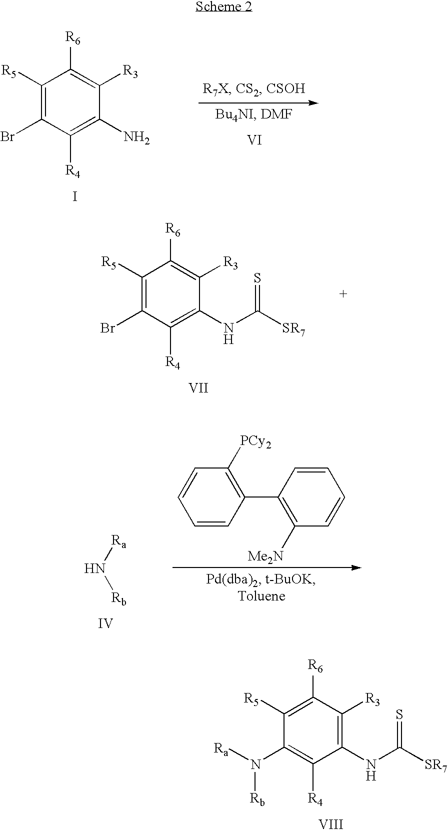

Section III. The preparation of compounds of formula XI is outlined in Scheme 3.

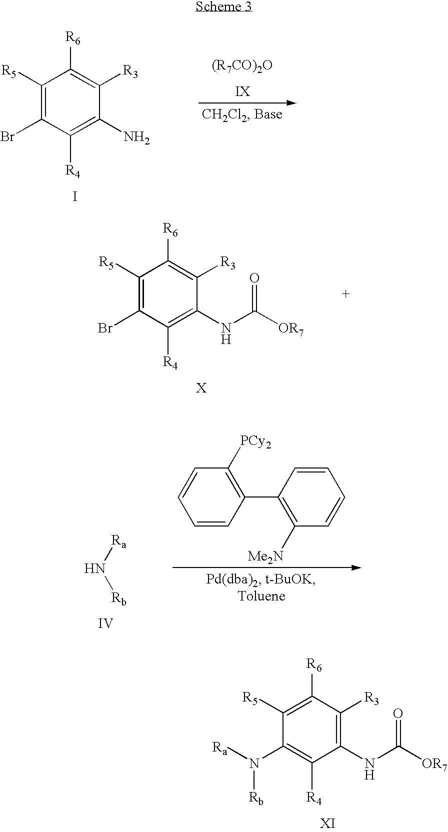

Section IV. The preparation of compounds of formula XII is outlined in Scheme 4.

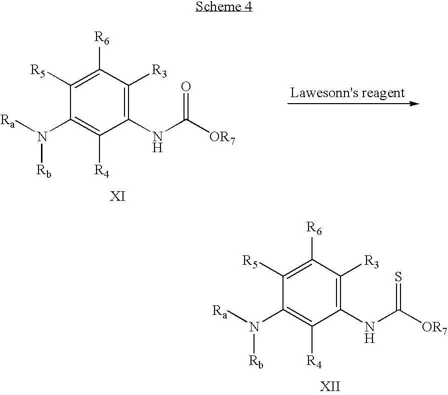

Section V. The preparation of compounds of formula VII is outlined in Scheme 5.

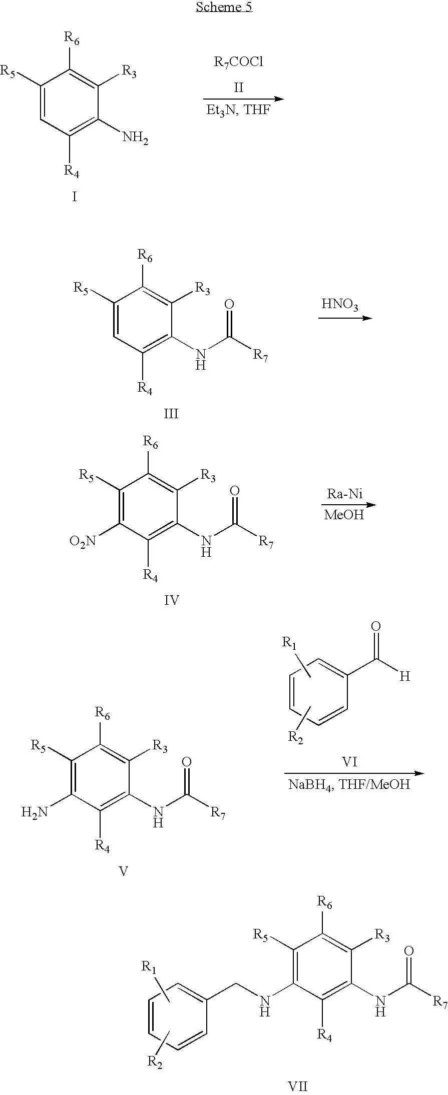

Section VI. An alternative preparation of compounds of formula VII is outlined in Scheme 6.

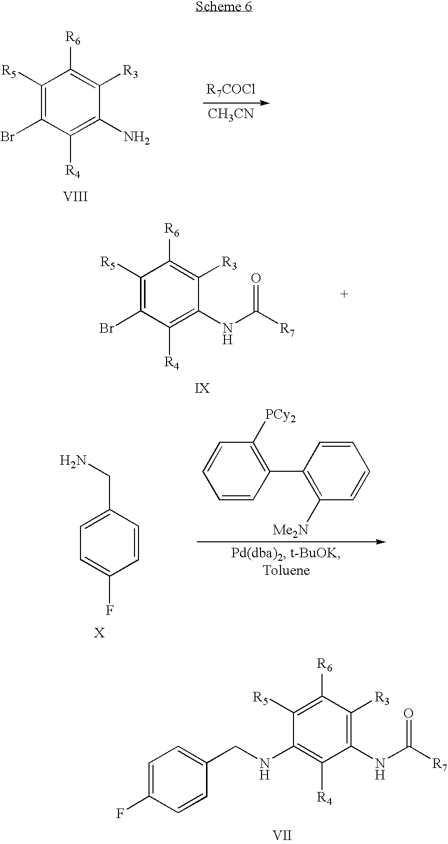

Section VII. The preparation of compounds of formula XIII is outlined in Scheme 7.

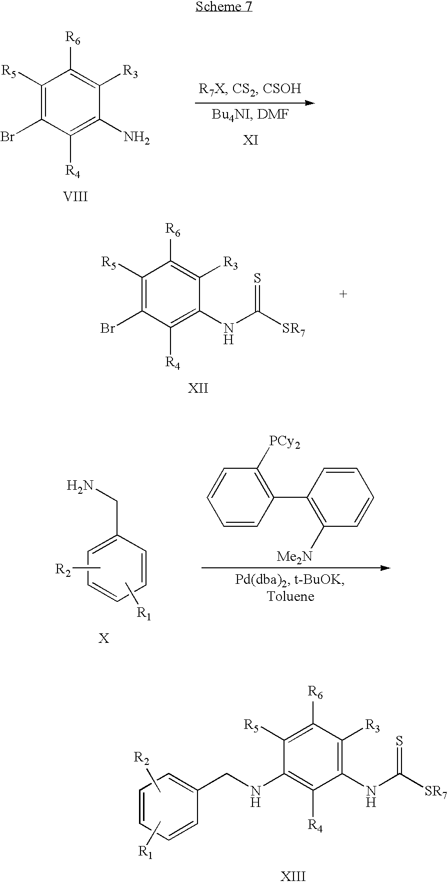

Section VIII. The preparation of compounds of formula XVI is outlined in Scheme 8.

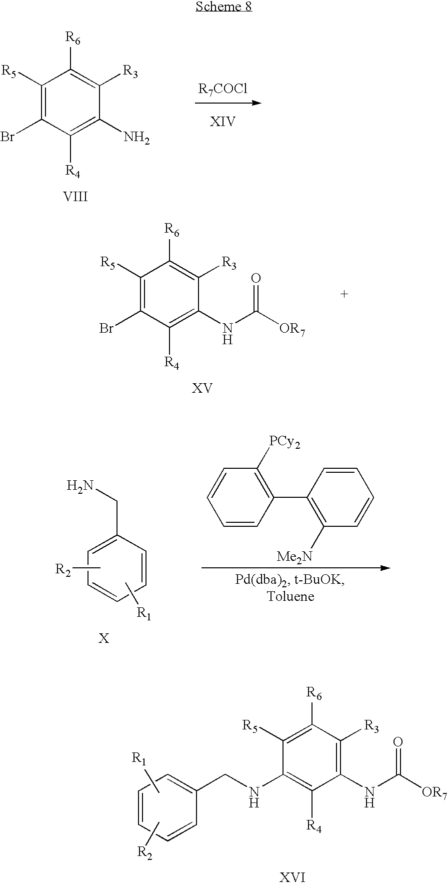

Section IX. The preparation of compounds of formula XVII is outlined in Scheme 9.

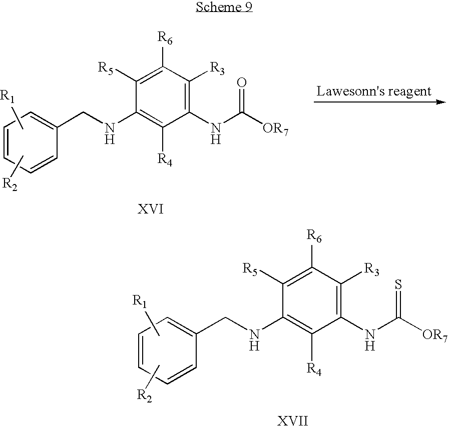

Section X. The preparation of compounds of formula XVIII is outlined in Scheme 10.

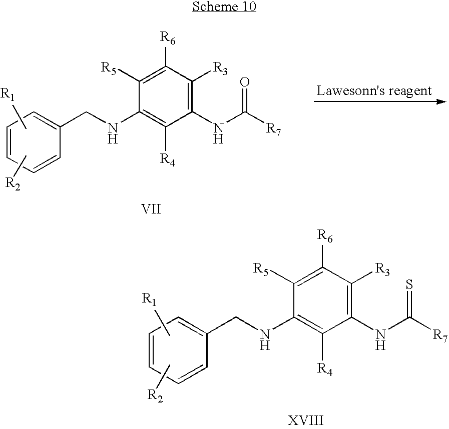

Section XI. The preparation of compounds of formula VI is outlined in Scheme 11

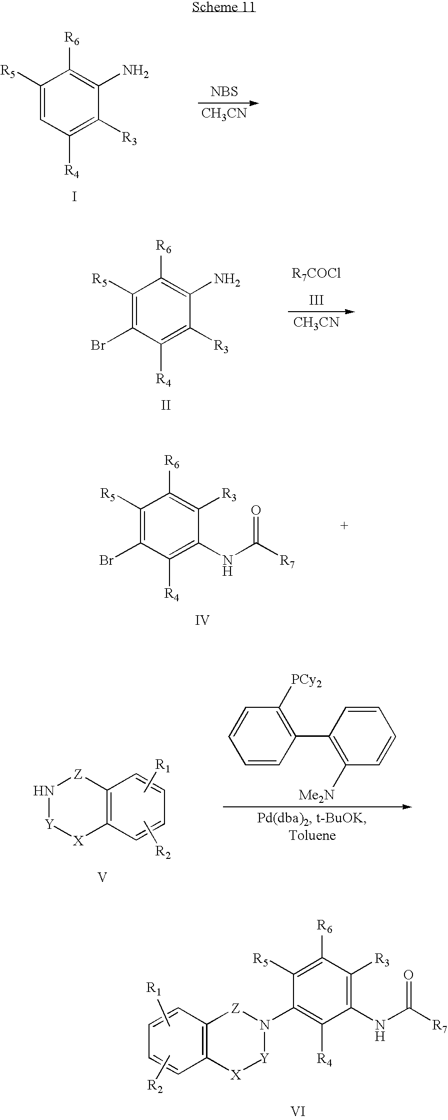

Section XII. The preparation of compounds of formula IX is outlined in Scheme 12.

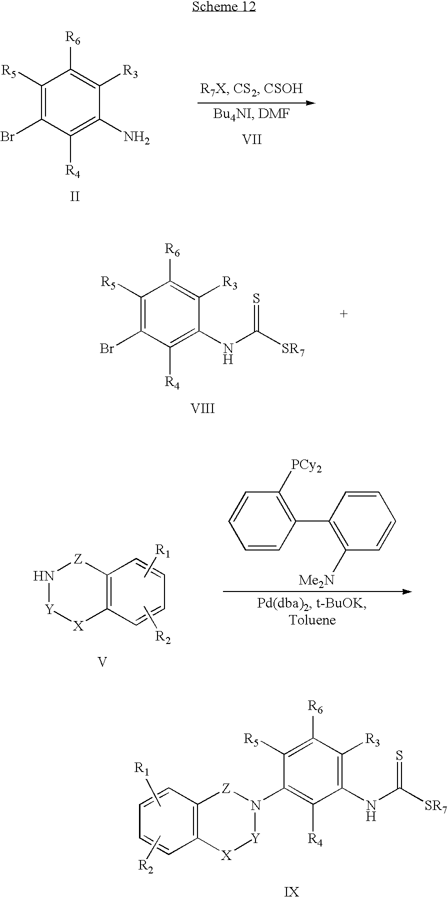

Section XIII. The preparation of compounds of formula XII is outlined in Scheme 13.

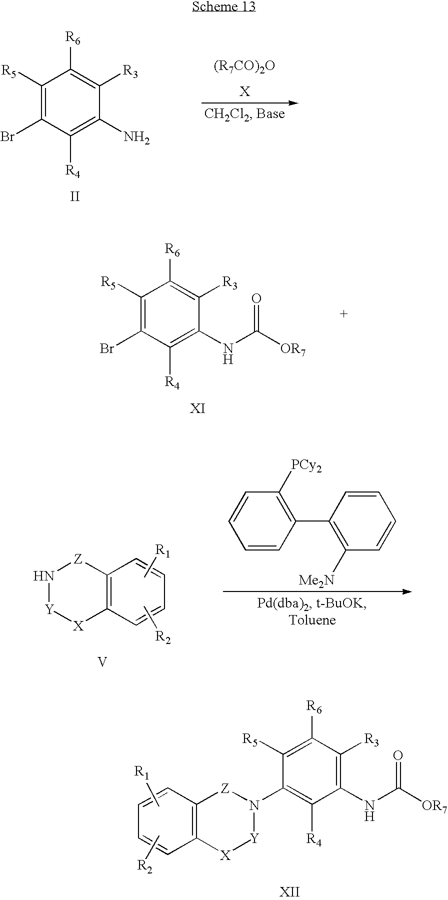

Section XIV. The preparation of compounds of formula XVII is outlined in Scheme 14.

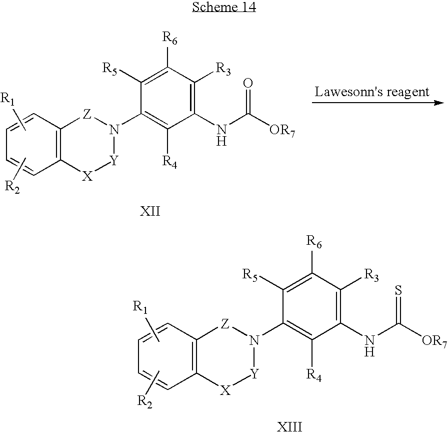

Section XV. The preparation of compounds of formula XIV is outlined in Scheme 15.

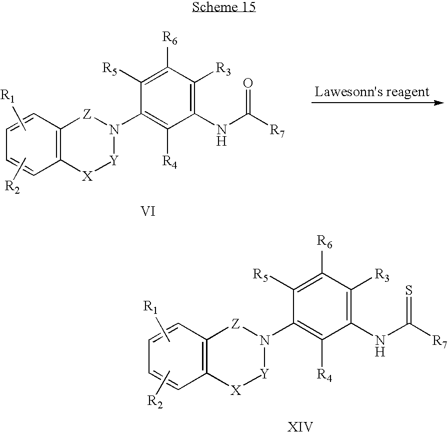

### EXAMPLES

### N-(5-(3,4-dihydroisoquinolin-2(1H)-yl)-2-methylphenyl)-3,3-dimethyl butanamide

### Step A: N-(5-bromo-2-methylphenyl)-3,3-dimethylbutanamide

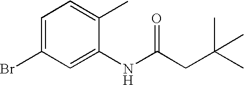

3,3-Dimethylbutanoyl chloride (1.0 g, 5.4 mmol) was added to a solution of 5-bromo-2-methylaniline (0.796 g, 5.9 mmol) in acetonitrile (10 mL). The reaction mixture was stirred at room temperature overnight. Water was added to the mixture and the precipitate formed collected to yield the title compound (0.9 g, 60%) as a white powder.

### Step B: N-(5-(3,4-dihydroisoquinolin-2(1H)-yl)-2-methylphenyl)-3,3-dimethylbutanamide

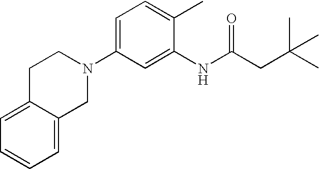

Bis(dibenzylidineacetone)palladium (2 mg, 0.0035 mmol) and (2′-dicyclohexylphosphanyl-biphenyl-2-yl)-dimethylamine (3.3 mg, 0.0084 mmol) were added to dry toluene (10 mL purged with argon) and stirred for 15 minutes under argon. Potassium tert-butoxide (157 mg, 1.4 mmol), 1,2,3,4-tetrahydroisoquinoline (113 mg, 0.85 mmol) and N-(5-bromo-2-methylphenyl)-3,3-dimethylbutanamide (200 mg, 0.7 mmol) were then added and the reaction mixture was stirred at 90° C. overnight. The reaction mixture was then cooled to room temperature, concentrated and purified by thin layer chromatography (Dichloromethane:Methanol 10%) to afford compound the title compound as a solid. (160 mg, 68%). ¹H NMR (DMSO-d₆, 300 MHz) δ 1.02 (s, 9H), 2.07 (s, 3H), 2.19 (s, 2H), 2.88 (t, J=5.4 Hz, 2H), 3.43(t, J=5.7 Hz, 2H), 4.27 (s, 2H), 6.76 (dd, J=8.4, 2.7 Hz, 1H), 7.03 (d, J=8.4 Hz, 2H), 7.19 (m, 4H), 9.06 (s, 1H).

The following compounds were prepared analogously:

### N-(2-fluoro-5-morpholinophenyl)-3,3-dimethylbutanamide

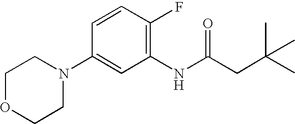

See example 1. Yield: 70%. ¹H NMR (DMSO-d₆, 300 MHz) δ 0.99 (s, 9H), 2.22 (s, 2H), 3.00 (t, J=4.8 Hz, 4H), 3.72 (t, J=4.5 Hz, 4H), 6.7(m, 1H), 7.09 (t, J=9.0 Hz, 1H), 7.40 (m, 1H), 9.43 (s, 1H).

### 3,3-Dimethyl-N-(2-methyl-5-morpholinophenyl)butanamide

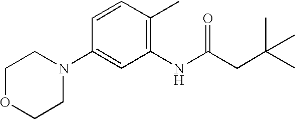

See example 1. Yield: 52%. ¹H NMR (CDCl₃, 300 MHz) δ 1.12 (s, 9H), 2.18 (s, 3H), 2.25 (s, 2H), 3.15 (t, J=4.8 Hz, 4H), 3.85 (t, J=4.8 Hz, 4H), 6.63 (dd, J=8.4, 2.4 Hz, 1H), 6.84 (s, 1H), 7.07 (d, J=8.4 Hz, 1H), 7.69 (d, J=2.1 Hz, 1H).

### 3,3-Dimethyl-N-(2-methyl-3-morpholinophenyl)butanamide

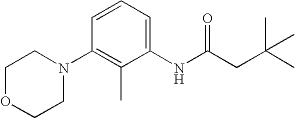

See example 1. Yield: 19%. ¹H NMR (CDCl₃, 300 MHz) δ 1.12 (s, 9H), 2.23 (s, 3H), 2.26 (s, 2H), 2.89 (t, J=4.5 Hz, 4H), 3.86 (t, J=4.5 Hz, 4H), 6.91 (d, J=8.1, Hz, 2H), 7.21 (t, J=8.1 Hz, 1H), 7.54 (d, J=8.1 Hz, 1H).

### N-(2,6-dimethyl-3-morpholinophenyl)-3,3-dimethylbutanamide

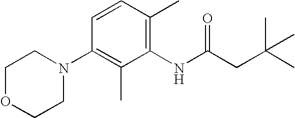

See example 1. Yield: 17%. ¹H NMR (CDCl₃, 300 MHz) δ 1.15 (s, 9H), 2.21 (s, 6H), 2.30 (s, 2H), 2.88 (t, J=4.8 Hz, 4H), 3.84 (t, J=4.8 Hz, 4H), 6.63 (s, 1H), 6.93 (d, J=8.4, Hz, 1H), 7.07 (d, J=8.1 Hz, 1H).

### 3-Cyclopentyl-N-(3-(4-fluorobenzylamino)-2,6-dimethylphenyl)propanamide

### Step A: 3-cyclopentyl-N-(2,6-dimethylphenyl)propanamide

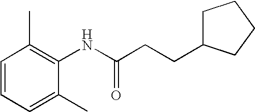

3-cyclopentylpropionyl chloride (2.166 g, 13 mmol) was added to a mixture of 2,6-dimethylaniline (1.211 g, 10 mmol) and triethylamine (1.515 g, 15 mmol) in THF at 0° C. The reaction mixture was progressively warmed to room temperature and stirred overnight. Ethyl acetate (200 ml) was added to the reaction mixture which was washed twice with HCl aq., 1N (100 ml). The organic layer was dried over MgSO₄ and concentrated. The oily crude product was subjected to crystallization form hexanes obtain the title compound (2.11 g, 8.6 mmol, 86%) as white crystal.

### Step B: 3-cyclopentyl-N-(2,6-dimethyl-3-nitrophenyl)propanamide

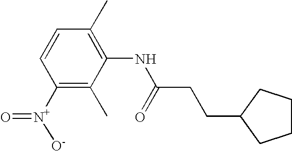

Fuming nitric acid (1.5 ml) was added dropwise to a mixture of 3-cyclopentyl-N-(2,6-dimethylphenyl)propanamide (1.0 g, 4.07 mmol) in acetic acid (3 ml) at 0° C. The reaction mixture was progressively warmed to room temperature and stirred for 2 hours and then poured onto cold water (50 ml). The mixture was extracted with ethyl acetate (100 ml). The organic layer was washed twice with NaOH aq., 1N (30 ml) and then dried over MgSO₄, concentrated and chromatographed (EtOAc:Hexanes, 2:3) to obtain the desired product (0.841 g, 2.896 mmol, 72%) as off-white powder.

### Step C: N-(3-amino-2,6-dimethylphenyl)-3-cyclopentylpropanamide

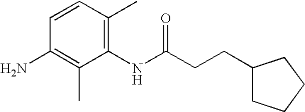

A suspension of Raney-Nickel (2 g) in a solution of 3-cyclopentyl-N-(2,6-dimethyl-3-nitrophenyl)propanamide (0.8 g, 2.75 mmol) in methanol (15 ml) and THF (5 ml) was vigorously stirred under hydrogen atmosphere (1 atm) at room temperature for 6 hours. The reaction mixture was filtered and then the filtrate was concentrated and chromatographed (EtOAc:Hexanes, 1:1) to obtain the title product (0.627 g, 2.4 mmol, 87%) as white powder.

### Step D: 3-cyclopentyl-N-(3-(4-fluorobenzylamino)-2,6-dimethylphenyl)propanamide

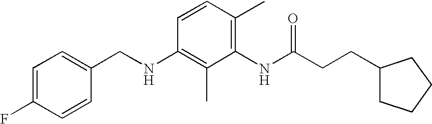

A mixture of N-(3-amino-2,6-dimethylphenyl)-3-cyclopentylpropanamide (0.226 g, 0.87 mmol) and 4-fluorobenzaldehyde (0.14 g, 1.13 mmol) in THF (3 ml) was stirred at room temperature for 3 hours and then NaBH₄ (0.15 g, 3.97 mmol) and MeOH (1 ml) were added simultaneously. The resulting mixture was stirred for an additional 2 hours. The mixture was extracted with ethyl acetate and washed twice with brine. The organic layer was dried over MgSO₄, concentrated, and chromatographed (EtOAc:Hexanes 1:3) to yield the title product (0.304 g, 0.825 mmol, 94%) as white powder. ¹H NMR (DMSO-d₆, 300 MHz) δ 1.00-1.17 (m, 2H), 1.40-1.62 (m, 6H), 1.70-1.81 (m, 3H), 1.93 (s, 6H), 2.28 (t, J=7.5 Hz, 2H), 4.27 (d, J=5.7 Hz, 2H), 5.47 (t, J=5.7 Hz, 1H), 6.17 (d, J=8.4 Hz, 1H), 6.70 (d, J=8.4 Hz, 1H), 7.08 (dd, J=8.7, 8.7 Hz, 2H), 7.35 (dd, J=8.3, 5.9 Hz, 2H), 9.05 (s, 1H).

### 3-cyclopentyl-N-(2,6-dimethyl-3-(4-(trifluoromethyl)benzylamino)phenyl)propanamide

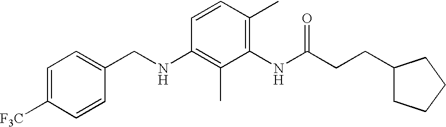

A solution of N-(3-amino-2,6-dimethylphenyl)-3-cyclopentylpropanamide (0.13 g, 0.5 mmol) and 4-(trifluoromethyl)benzaldehyde (0.122 g, 0.7 mmol) in THF (3 ml) was stirred at room temperature for 3 hours and then NaBH₄ (0.106 g, 2.8 mmol) and MeOH (1 ml) were added simultaneously. The resulting mixture was stirred for an additional 12 hours. The mixture was extracted with ethyl acetate and washed twice with brine. The organic layer was dried over MgSO₄, concentrated and chromatographed (EtOAc:Hexanes, 1:3) to obtain the title compound (0.097 g, 0.232 mmol, 46%) as white powder. ¹H NMR (DMSO-d₆, 500 MHz) δ 1.05-1.22 (m, 2H), 1.40-1.52 (m, 2H), 1.55-1.65 (m, 4H), 1.70-1.82 (m, 3H), 1.93 (s, 3H), 1.95 (s, 3H), 2.27 (t, J=7.5 Hz, 2H), 4.40 (d, J=5.7 Hz, 2H), 5.62 (t, J=5.7 Hz, 1H), 6.13 (d, J=8.2 Hz, 1H), 6.96 (d, J=8.2 Hz, 1H), 7.52 (d, J=7.8 Hz, 2H), 7.63 (d, J=7.8 Hz, 2H), 9.05 (s, 1H).

### N-(6-ethyl-3-(4-fluorobenzylamino)-2-methylphenyl)-3,3-dimethylbutanamide

### Step A: N-(2-ethyl-6-methylphenyl-3,3-dimethylbutanamide

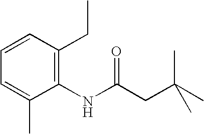

ter-Butylacetyl chloride (3.63 ml, 26 mmol) was added to a mixture of 6-ethyl-o-toluidine (2.712 g, 20 mmol) and triethylamine (4.17 ml, 30 mmol) in THF at 0° C. The reaction mixture was progressively warmed to room temperature and stirred overnight. Ethyl acetate (200 ml) was added to the reaction mixture which was washed twice with HCl aq., 1N (100 ml). The organic layer was dried over MgSO₄ and concentrated. The oily crude product was subjected to crystallization form hexanes to yield the title compound (3.982 g, 17 mmol, 85%) as white needle crystal.

### Step B: N-(6-ethyl-2-methyl-3-nitrophenyl)-3,3-dimethylbutanamide and N-(2-ethyl-6-methyl-3-nitrophenyl)-3,3-dimethylbutanamide

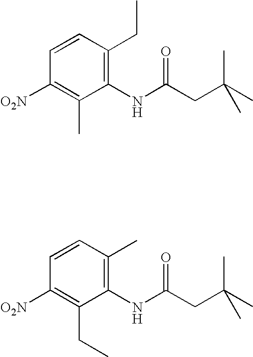

Fuming nitric acid (3 ml) was added dropwise to a mixture of N-(2-ethyl-6-methylphenyl)-3,3-dimethylbutanamide_(1.88 g, 8 mmol) in acetic acid (5 ml) at 0° C. The reaction mixture was progressively warmed to room temperature and stirred for 12 hours and then poured onto cold water (50 ml). The mixture was extracted with ethyl acetate (100 ml). The organic layer was washed twice with NaOH aq., 1N (30 ml), dried over MgSO₄, concentrated and chromatographed (EtOAc:Hexanes, 1:3) to yield N-(6-ethyl-2-methyl-3-nitrophenyl)-3,3-dimethylbutanamide (0.654 g, 2.35 mmol, 29%) as off-white powder and N-(2-ethyl-6-methyl-3-nitrophenyl)-3,3-dimethylbutanamide (0.258 g, 0.93 mmol, 11%) as off-white powder.

### Step C: N-(3-amino-6-ethyl-2-methylphenyl)-3,3-dimethylbutanamide

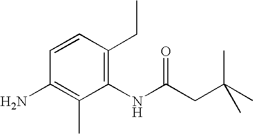

A suspension of Raney-Nickel (1 g) in a solution of N-(6-ethyl-2-methyl-3-nitrophenyl)-3,3-dimethylbutanamide (0.654 g, 2.35 ml) in methanol (20 ml) was vigorously stirred under hydrogen atmosphere (1 atm) at room temperature for 3 hours. The reaction mixture was filtered and then the filtrate was concentrated and chromatographed (EtOAc:Hexanes, 1:1) to yield to N-(6-ethyl-3-(4-fluorobenzylamino)-2-methylphenyl)-3,3-dimethylbutanamide ( 0.358 g, 1.44 mmol, 61%) as white powder.

### Step D: N-(6-ethyl-3-(4-fluorobenzylamino)-2-methylphenyl)-3,3-dimethylbutanamide

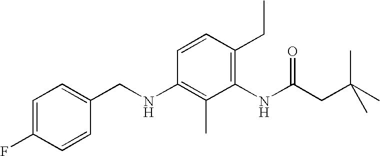

A mixture of N-(3-amino-6-ethyl-2-methylphenyl)-3,3-dimethylbutanamide (0.2 g, 0.8 mmol) and 4-fluorobenzaldehyde (0.149 g, 1.2 mmol) in THF (5 ml) was stirred at room temperature for 3 hours and then NaBH₄ (0.151 g, 4.0 mmol) and MeOH (1 ml) were added into the mixture simultaneously. The mixture was stirred for an additional 3 hours. The mixture was extracted with ethyl acetate and washed twice with brine. The organic layer was dried over MgSO₄, concentrated and purified by chromatography on silica (EtOAc:Hexanes, 1:3) to yield to the desired product (0.183 g, 0.514 mmol, 64%) as white powder. ¹H NMR (DMSO-d₆, 500 MHz) δ 0.97 (t, J=7.5 Hz, 3H), 1.05 (s, 9H), 1.95 (s, 3H), 2.19 (s, 2H), 2.32 (q, J=7.5 Hz, 2H), 4.24 (d, J=5.5 Hz, 2H), 5.51 (t, J=5.5 Hz, 1H), 6.24 (d, J=8.2 Hz, 1H), 6.73 (d, J=8.2 Hz, 1H), 7.10 (dd, J=8.8, 8.8 Hz, 2H), 7.37 (dd, J=8.3, 5.9 Hz, 2H), 8.96 (s, 1H).

### N-(2-ethyl-3-(4-fluorobenzylamino)-6-methylphenyl)-3,3-dimethylbutanamide

### Step A: N-(3-amino-2-ethyl-6-methylphenyl)-3,3-dimethylbutanamide

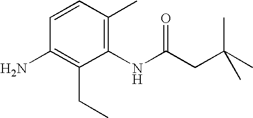

A suspension of Raney-Nickel (1 g) in a solution of N-(2-ethyl-6-methyl-3-nitrophenyl)-3,3-dimethylbutanamide (see example 4, 0.258 g, 0.93 mmol) in methanol (15 ml) was vigorously stirred under hydrogen atmosphere (1 atm) at room temperature for 3 hours. The reaction mixture was filtered and then the filtrate was concentrated and chromatographed (EtOAc:Hexanes, 1:1) to yield to the title product (0.189 g, 0.76 mmol, 81%) as off-white powder.

### Step B: N-(2-ethyl-3-(4-fluorobenzylamino)-6-methylphenyl)-3,3-dimethylbutanamide

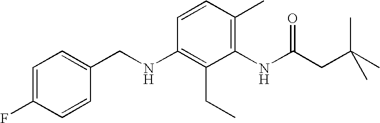

A solution of N-(3-amino-2-ethyl-6-methylphenyl)-3,3-dimethylbutanamide (0.046 g, 0.185 mmol) and 4-fluorobenzaldehyde (0.03 g, 2.4 mmol) in THF (2 ml) was stirred at room temperature for 3 hours and then NaBH₄ (0.038 g, 1.0 mmol) and MeOH (0.5 ml) were added simultaneously. The resulting mixture was stirred for an additional 3 hours. The mixture was extracted with ethyl acetate and washed twice with brine. The organic layer was dried over MgSO₄, concentrated and chromatographed (EtOAc:Hexanes, 1:3) to yield the title product (0.035 g, 0.098 mmol, 53%) as white powder. ¹H NMR (CDCl₃, 500 MHz) δ 0.92 (t, J=7.5 Hz, 3H), 1.13 (s, 9H), 2.03 (s, 3H), 2.40 (s, 2H), 2.42 (q, J=7.5 Hz, 2H), 4.15 (s, 2H), 6.91 (d, J=8.2 Hz, 1H), 6.98-7.09 (m, 3H), 7.23=7.27 (m, 2H), 7.45 (s, 1H). (rotomer effect)

### N-(3-(4-fluorobenzylamino)-2,6-dimethylphenyl)-3,3-dimethylbutanamide

### Step A: N-(2,6-dimethylphenyl)-3,3-dimethylbutanamide

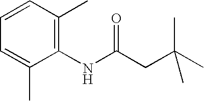

T-butyl-acetyl chloride (1.83 ml, 13 mmol) was added to a mixture of 2,6-dimethylaniline (1.21 g, 10 mmol) and triethylamine ( 1.8 ml, 13 mmol) in THF at 0° C. The reaction mixture was progressively warmed to room temperature and stirred overnight. Ethyl acetate (200 ml) was added to the reaction mixture which was washed twice with HCl aq., 1N (100 ml). The organic layer was dried over MgSO₄ and concentrated. The oily crude product was subjected to crystallization form hexanes to yield the title compound (1.805 g, 8.24 mmol, 82%) as white crystal.

### Step B: N-(2,6-dimethyl-3-nitrophenyl)-3,3-dimethylbutanamide

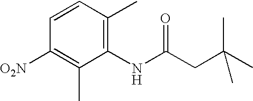

Fuming nitric acid (3 ml) was added dropwise to a mixture of N-(2,6-dimethylphenyl)-3,3-dimethylbutanamide (1.524 g, 6.95 mmol) in acetic acid (4 ml) at 0° C. The reaction mixture was progressively warmed to room temperature and stirred for 2 hours and then poured onto cold water (50 ml). The mixture was extracted with ethyl acetate (100 ml). The organic layer was washed twice with NaOH aq., 1N (30 ml) and then dried over MgSO₄, concentrated and chromatographed (EtOAc:Hexanes, 1:3) to yield to the title compound (1.62 g, 6.13 mmol, 88%) as off-white powder.

### Step C: N-(3-amino-2,6-dimethylphenyl)-3,3-dimethylbutanamide

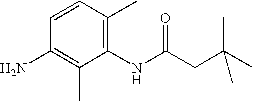

A suspension of Raney-Nickel (1 g) in a solution of N-(2,6-dimethyl-3-nitrophenyl)-3,3-dimethylbutanamide (1.06 g, 4.0 ml) in methanol (20 ml) was vigorously stirred under hydrogen atmosphere (1 atm) at room temperature for 12 hours. The reaction mixture was filtered and then the filtrate was concentrated and chromatographed (EtOAc:Hexanes, 3:1) to yield to the title compound (0.664 g, 2.84 mmol, 71%) as white powder.

### Step D: N-(3-(4-fluorobenzylamino)-2,6-dimethylphenyl)-3,3-dimethylbutanamide

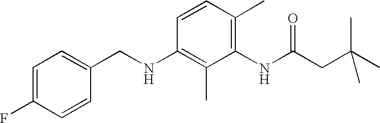

A solution of N-(3-amino-2,6-dimethylphenyl)-3,3-dimethylbutanamide (0.327 g, 1.4 mmol) and 4-fluorobenzaldehyde (0.226 g, 1.82 mmol) in THF (3 ml) was stirred at room temperature for 2 hours and then NaBH₄ (0.211 g, 5.6 mmol) and MeOH (1 ml) were added simultaneously. The resulting mixture was stirred for an additional 2 hours. The mixture was extracted with ethyl acetate and washed twice with brine. The organic layer was dried over MgSO₄, concentrated and chromatographed (EtOAc:Hexanes, 1:3) to yield to the title compound (0.271 g, 0.792 mmol, 56%) as white powder (0.113 g, 0.33 mmol, 24%) as white powder. ¹H NMR (DMSO-d₆, 500 MHz) δ 1.06 (s, 9H), 1.98 (s, 6H), 2.20 (s, 2H), 4.31 (d, J=5.7 Hz, 2H), 5.52 (t, J=5.7 Hz, 1H), 6.21 (d, J=8.2 Hz, 1H), 6.73 (d, J=8.2 Hz, 1H), 7.10 (dd, J=8.8, 8.8 Hz, 2H), 7.37 (dd, J=8.3, 5.9 Hz, 2H), 9.01 (s, 1H).

### N-(2,6-dimethyl-3-(4-(trifluoromethyl)benzylamino)phenyl)-3,3-dimethylbutanamide

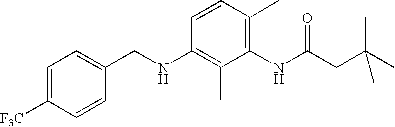

A solution of N-(3-amino-2,6-dimethylphenyl)-3,3-dimethylbutanamide (0.327 g, 1.4 mmol) and 4-(trifluoromethyl)benzaldehyde (0.337 g, 1.82 mmol) in THF (5 ml) was stirred in the presence of MS 4A at room temperature for 3 hours and then NaBH₄ (0.2.11 g, 5.6 mmol) and MeOH (0.5 ml) were added simultaneously. The resulting mixture was stirred for an additional 24 hours. The mixture was extracted with ethyl acetate and washed twice with brine. The organic layer was dried over MgSO₄, concentrated and chromatographed (EtOAc:Hexanes, 1:3) to yield to the title compound (0.159 g, 0.4 mmol, 28%) as white powder. ¹H NMR (DMSO-d₆, 500 MHz) δ 1.07 (s, 9H), 1.98 (s, 3H), 2.00 (s, 3H), 2.21 (s, 2H), 4.43 (d, J=5.7 Hz, 2H), 5.66 (t, J=5.7 Hz, 1H), 6.16 (d, J=8.2 Hz, 1H), 6.73 (d, J=8.2 Hz, 1H), 7.56 (d, J=8.3 Hz, 2H), 7.67 (d, J=8.3 Hz, 2H), 9.03 (s, 1H).

### 3-Cyclopentyl-N-(2,6-diethyl-3-(4-fluorobenzylamino)phenyl)propanamide

### Step A: 3-cyclopentyl-N-(2,6-diethylphenyl)propanamide

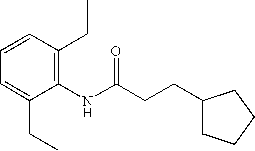

3-Cyclopentylpropionyl chloride (3.25 g, 19.5 mmol) was added to a mixture of 2,6-diethylaniline (2.239 g, 15 mmol) and triethylamine (2.02 g, 20 mmol) in THF at 0° C. The reaction mixture was progressively warmed to room temperature and stirred overnight. Ethyl acetate (200 ml) was added to the reaction mixture which was washed twice with HCl aq., 1N (100 ml). The organic layer was dried over MgSO₄ and concentrated. The oily crude product was subjected to crystallization form hexanes to yield to the title compound (3.74 g, 13.4 mmol, 89%) as white crystal.

### Step B: 3-cyclopentyl-N-(2,6-diethyl-3-nitrophenyl)propanamide

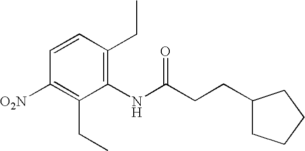

Fuming nitric acid (5.0 ml) was added dropwise to a mixture of 3-cyclopentyl-N-(2,6-diethylphenyl)propanamide (3.74 g, 13.4 mmol) in acetic acid (10 ml) at 0° C. The reaction mixture was progressively warmed to room temperature and stirred for 5 hours and then poured onto cold water (100 ml). The mixture was extracted with ethyl acetate (200 ml). The organic layer was washed twice with NaOH aq., 1N (50 ml) and then dried over MgSO₄, concentrated and chromatographed (EtOAc:Hexanes, 2:3) to yield the title compound (2.515 g, 7.9 mmol, 59%) as off-white powder.

### Step C: N-(3-amino-2,6-diethylphenyl)-3-cyclopentylpropanamide

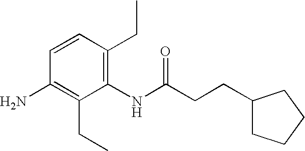

A suspension of Raney-Nickel (1 g) in a solution of 3-cyclopentyl-N-(2,6-diethyl-3-nitrophenyl)propanamide (1.0 g, 3.0 mmol) in methanol (15 ml) was vigorously stirred under hydrogen atmosphere (1 atm) at room temperature for 12 hours. The reaction mixture was filtered and then the filtrate was concentrated and chromatographed (EtOAc:Hexanes, 1:1) to yield the title compound (0.774 g, 2.63 mmol, 88%) as white powder.

### Step D: 3-cyclopentyl-N-(2,6-diethyl-3-(4-fluorobenzylamino)phenyl)propanamide

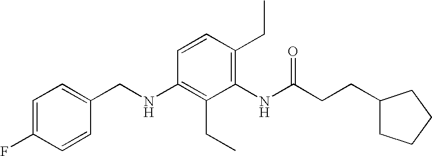

A mixture of N-(3-amino-2,6-diethylphenyl)-3-cyclopentylpropanamide (0.383 g, 1.3 mmol) and 4-fluorobenzaldehyde (0.242 g, 1.95 mmol) in THF (4 ml) was stirred at room temperature for 3 hours and then NaBH₄ (6.5 g, 0245 mmol) and MeOH (1 ml) were added simultaneously. The resulting mixture was stirred for an additional 2 hours. The mixture was extracted with ethyl acetate and washed twice with brine. The organic layer was dried over MgSO₄, concentrated and chromatographed (EtOAc:Hexanes, 1:3) to yield to the title compound (0.195 g, 0.492 mmol, 38%) as white powder. ¹H NMR (DMSO-d₆, 500 MHz) δ 0.97 (t, J=7.5 Hz, 3H), 1.01 (t, J=7.5 Hz, 3H), 1.04-1.10 (m, 2H), 1.40-1.50 (m, 2H), 1.55-1.65 (m, 4H), 1.70-1.82 (m, 3H), 1.93 (s, 6H), 2.28 (q, J=7.5 Hz, 4H), 2.35-2.45 (m, 2H), 4.29 (s, 2H), 5.67 (bs, 1H), 6.23 (d, J=8.2 Hz, 1H), 6.72 (d, J=8.2 Hz, 1H), 7.10 (dd, J=8.8, 8.8 Hz, 2H), 7.37 (dd, J=8.3, 5.8 Hz, 2H), 8.99 (s, 1H).

### 2-Cyclopentyl-N-(3-(4-fluorobenzylamino)-6-methoxy-2-methylphenyl)acetamide

### Step A: 2-cyclopentyl-N-(2-methoxy-6-methylphenyl)acetamide

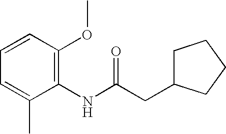

3-cyclopentylpropionyl chloride (2.24 g, 15.31 mmol) was added to a mixture of 2-methoxy-6-methylaniline (2.0 g, 14.58 mmol) and triethylamine (2.24 mL, 16.04 mmol) in DCM at 0° C. The reaction mixture was progressively warmed to room temperature and stirred overnight. Ethyl acetate (200 ml) was added to the reaction mixture which was washed twice with HCl aq., 1N (100 ml). The organic layer was dried over MgSO₄ and concentrated. The oily crude product was subjected to crystallization form hexanes to yield to the title compound (3.55 g, 14.37 mmol, 99%) as white crystal.

### Step B: 2-cyclopentyl-N-(6-methoxy-2-methyl-3-nitrophenyl)acetamide

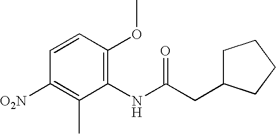

Fuming nitric acid (2 ml) was added dropwise to a mixture of 2-cyclopentyl-N-(2-methoxy-6-methylphenyl)acetamide (1.0 g, 4.04 mmol) in acetic acid (3 ml) at 0° C. The reaction mixture was progressively warmed to room temperature and stirred for 2 hours and then poured onto cold water (50 ml). The mixture was extracted with ethyl acetate (100 ml). The organic layer was washed twice with NaOH aq., 1N (30 ml) and then dried over MgSO₄, concentrated and isolated by column chromatography on silica gel (EtOAc:Hexanes, 2:3) to yield to the desired product (0.505 g, 1.73 mmol, 43%) as a mixture of two isomers.

### Step C: N-(3-amino-6-methoxy-2-methylphenyl)-2-cyclopentylacetamide

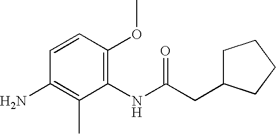

A suspension of Raney-Nickel (100 mg) in a solution of 2-cyclopentyl-N-(6-methoxy-2-methyl-3-nitrophenyl)acetamide (0.8 g, 2.75 mmol) in ethanol (10 ml) was vigorously stirred under hydrogen atmosphere (1 atm) at room temperature for 6 hours. The reaction mixture was filtered and then the filtrate was concentrated and isolated by column chromatography on silica gel (EtOAc:Hexanes, 1:1) to yield to the desired compound (0.385 g, 1.47 mmol, 85%) as a mixture of two isomers.

### Step D: 2-cyclopentyl-N-(3-(4-fluorobenzylamino)-6-methoxy-2-methylphenyl)acetamide

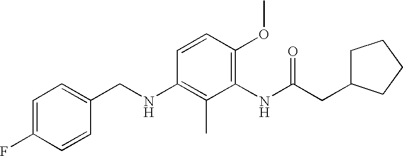

A mixture of the previous reaction (0.385 g, 1.47 mmol) and 4-fluorobenzaldehyde (0.24 mL, 2.21 mmol) in THF (3 ml) were stirred at room temperature for 3 hours and then NaBH₄ (0.280 mg, 7.35 mmol) and MeOH (1 ml) were added simultaneously. The resulting mixture was stirred for an additional 2 hours. The mixture was extracted with ethyl acetate and washed twice with brine. The organic layer was dried over MgSO₄, concentrated and isolated by column chromatography on silica gel (EtOAc:Hexanes, 1:3) to yield the title compound (0.165 g, 0.73 mmol, 30%) as off-white powder. ¹H NMR (CDCl₃, 300 MHz) δ 1.23-1.30 (m, 2H), 1.60-1.70 (m, 4H), 1.89-1.96 (m, 2H), 2.03 (s, 3H), 2.32-2.37 (m, 1H), 2.43 (d, J=6.3 Hz, 2H), 3.64 (bs, 1H), 3.72 (s, 3H), 4.28 (s, 2H), 6.44 (d, J=9.0 Hz, 1H), 6.66 (d, J=8.7, Hz, 1H), 6.95 (s, 1H), 7.05 (t, J=8.7 Hz, 2H), 7.36 (dd, J=8.1, 5.4 Hz, 2H).

### N-(6-chloro-3-(4-fluorobenzylamino)-2-methylphenyl)-3,3-dimethyl butanamide

### Step A: 3-bromo-6-chloro-2-methylaniline

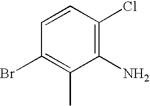

N-chlorosuccinimide ( 0.359 g, 2.69 mmol) was added to a solution of 3-bromo-2-methyl aniline (0.5 g, 2.69 mmol) in acetonitrile (5 mL) at room temperature. The reaction mixture was then stirred at 60° C. for 24 hours, cooled to room temperature and concentrated. Purification by column chromatography (5-20% EtOAc:Hexanes) afforded compound 1a (0.135 g, 0.61 mmol) in 23% yield.

### Step B: N-(3-bromo-6-chloro-2-methylphenyl)-3,3-dimethylbutanamide and N-(3-bromo-4-chloro-2-methylphenyl-3,3-dimethylbutanamide

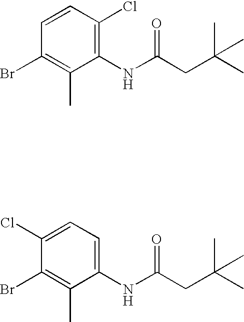

3,3-Dimethylbutanoyl chloride (0.085 mL, 0.61 mmol) was added to a mixture 3-bromo-6-chloro-2-methylaniline (0.135 g, 0.61 mmol) and triethylamine (0.09 mL, 0.64 mmol) in DCM (2.5 mL) at 0° C. The reaction mixture was then stirred at 50° C. overnight. Ethyl acetate (50 ml) was added to the reaction mixture which was washed twice with HCl aq., 1N (20 ml). The organic layer was dried over MgSO₄ and concentrated. Purification by column chromatography (15-30% EtOAc:Hexanes) afforded N-(3-bromo-6-chloro-2-methylphenyl)-3,3-dimethylbutanamide and N-(3-bromo-4-chloro-2-methylphenyl)-3,3-dimethylbutanamide as white solids.

### Step C: N-(6-chloro-3-(4-fluorobenzylamino)-2-methylphenyl)-3,3-dimethylbutanamide

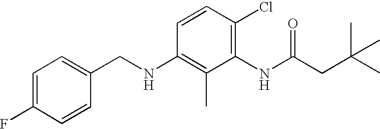

Bis(dibenzylidineacetone)palladium (13 mg, 0.022 mmol) and (2′-dicyclohexylphosphanyl-biphenyl-2-yl)-dimethylamine (14 mg, 0.035 mmol) were added to dry toluene (1.5 mL purged with argon) and stirred for 15 minutes under argon. Potassium tert-butoxide (74 mg, 0.66 mmol), 4-fluorobenzyl amine (100 μL, 0.91 mmol) and N-(6-chloro-3-(4-fluorobenzylamino)-2-methylphenyl)-3,3-dimethylbutanamide (100 mg, 0.314 mmol) were then added and the reaction mixture was stirred at 90° C. overnight. The reaction mixture was then cooled to room temperature, concentrated and purified by thin layer chromatography (Dichloromethane:Methanol 10%) to afford compound 1c as a solid. (67 mg, 60% yield). ¹H NMR (CDCl₃, 300 MHz) δ 1.15 (s, 9H), 2.05 (s, 3H), 2.32 (s, 2H), 3.88 (bs, 1H), 4.31 (s, 2H), 6.44 (d, J=9.0 Hz, 1H), 6.92 (s, 1H), 7.06 (t, J=8.4, Hz, 2H), 7.12 (d, J=9 Hz, 1H), 7.34 (dd, J=8.7, 5.4 Hz, 2H).

### N-(4-chloro-3-(4-fluorobenzylamino)-2-methylphenyl)-3,3-dimethyl butanamide

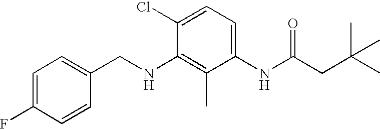

This compound was prepared analogously (example 10) from N-(3-bromo-4-chloro-2-methylphenyl)-3,3-dimethylbutanamide (step B, example 10) and 4-fluorobenzyl amine. Yield: 53% (Step C). ¹H NMR (CDCl₃, 300 MHz) δ 1.12 (s, 9H), 2.56 (s, 3H), 2.27 (s, 2H), 3.8 (bs, 1H), 4.07 (d, J=5.7 Hz, 2H), 6.84 (m, 1H), 7.05 (m, 2H), 7.17 (m, 1H), 7.34 (m, 2H).

### N-(2-chloro-3-(4-fluorobenzylamino)-6-methylphenyl)-3,3-dimethyl butanamide

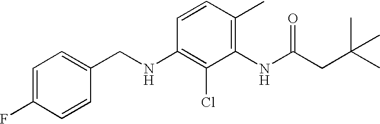

This compound was prepared analogously (example 10) from N-(3-bromo-2-chloro-6-methylphenyl)-3,3-dimethylbutanamide and 4-fluorobenzyl amine. Yield: 62% (Step C). ¹H NMR (CDCl₃, 300 MHz) δ 1.15 (s, 9H), 2.16 (s, 3H), 2.31 (s, 2H), 4.35 (d, J=5.7 Hz, 2H), 4.60 (bs, 1H), 6.48 (d, J=8.1 Hz, 1H), 6.79 (bs, 1H), 6.98 (d, J=8.7, Hz, 1H), 7.05 (t, J=8.4 Hz, 2H), 7.33 (dd, J=8.7, 5.7 Hz, 2H).

### N-(4-chloro-5-(4-fluorobenzylamino)-2-methylphenyl)-3,3-dimethyl butanamide

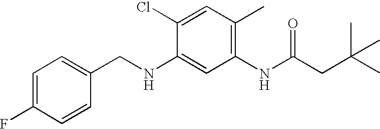

This compound was prepared analogously (example 10) from N-(3-bromo-2-chloro-6-methylphenyl)-3,3-dimethylbutanamide and 4-fluorobenzyl amine. Yield: 37% (Step C). ¹H NMR (CDCl₃, 300 MHz) δ 1.09 (s, 9H), 2.12 (s, 3H), 2.21 (s, 2H), 4.34 (s, 2H), 6.75 (m,1H), 7.04 (m, 2H), 7.36(m, 3H).

### N-(5-(4-fluorobenzylamino)-2-methylphenyl)-3,3-dimethylbutanamide

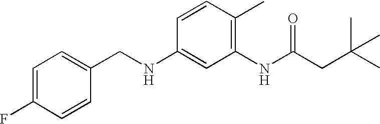

This compound was prepared analogously (example 10, Step C) from N-(5-bromo-2-methylphenyl)-3,3-dimethylbutanamide and 4-fluorobenzyl amine. Yield: 35%. ¹H NMR (CDCl₃, 300 MHz) δ 1.03 (s, 9H), 2.17 (s, 3H), 2.26 (s, 2H), 4.29 (bs, 2H), 4.43 (bs, 1H), 6.65 (dd, J=8.1, 2.1 Hz, 1H), 7.01 (q, J=7.8, Hz, 2H), 7.20 (s, 1H), 7.29 (m, 3H), 7.42 (s, 1H).

### N-(3-(4-fluorobenzylamino)-2-methylphenyl)-3,3-dimethylbutanamide

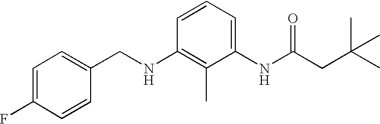

This compound was prepared analogously (example 10, Step C) from N-(3-bromo-2-methylphenyl)-3,3-dimethylbutanamide and 4-fluorobenzyl amine Yield: 57%. ¹H NMR (CDCl₃, 300 MHz) δ 1.13 (s, 9H), 2.04 (s, 3H), 2.25 (s, 2H), 3.89 (bs, 1H), 4.34 (d, J=4.2 Hz, 2H), 6.46 (d, J=7.8, Hz, 1H), 6.88 (d, J=7.2, Hz, 2H), 7.09 (q, J=8.7 Hz, 3H), 7.35 (m, 2H).

### N-(2,4-difluoro-5-(4-fluorobenzylamino)phenyl)-3,3-dimethylbutanamide

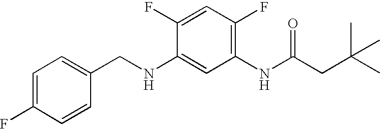

This compound was prepared analogously (example 10, Step C) from N-(5-bromo-2,4-difluorophenyl)-3,3-dimethylbutanamide (example 10, Step B) and 4-fluorobenzyl amine Yield: 20%. ¹H NMR (CDCl₃, 300 MHz) δ 1.09 (s, 9H), 2.23 (s, 2H), 3.89 (bs, 1H), 4.30 (d, J=4.2 Hz, 2H), 6.85 (t, 1H), 7.05 (m, 3H), 7.34 (m, 2H), 7.35 (t, 1H).

### N-(4-fluoro-3-(4-fluorobenzylamino)phenyl)-3,3-dimethylbutanamide

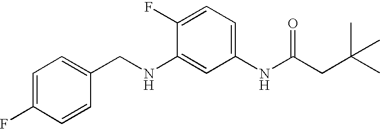

This compound was prepared by a method analogous to the synthesis of example 10, Step C, from N-(3-bromo-4-fluorophenyl)-3,3-dimethylbutanamide (see example 10, Step B) and 4-fluorobenzyl amine Yield: 47%. ¹H NMR (CDCl₃, 300 MHz) δ 1.08 (s, 9H), 2.17 (s, 2H), 4.26 (bs, 1H), 4.33 (d, J=4.2 Hz, 2H), 6.63 (m, 1H), 6.89 (m, 2H), 7.03 (m, 3H), 7.34 (m, 1H).

Compounds of formulas IB, IC, ID, IE, IF, IG, and IJ can be prepared by analogous reaction schemes, using the appropriate amine precursors in Schemes 1 and 2.

### Biological Results

Compounds of this invention formula were evaluated as KCNQ 2/3 modulators by measuring rhubidium release in the following assay.

Methods: PC-12 cells were grown at 37° C. and 5% CO₂ in DMEM/F12 Medium supplemented with 10% horse serum, 5% fetal bovine serum, 2 mM glutamine, 100 U/ml penicillin, 100 U/ml streptomycin. They were plated in poly-D-lysine-coated 96-well cell culture microplates at a density of 40,000 cells/well and differentiated with 100 ng/ml NGF-7s for 2-5 days. For the assay, the medium was aspirated and the cells were washed once with 0.2 ml in wash buffer (25 mM Hepes, pH 7.4, 150 mM NaCl, 1 mM MgCl₂, 0.8 mM NaH₂PO₄, 2 mM CaCl₂). The cells were then loaded with 0.2 ml Rb⁺ loading buffer (wash buffer plus 5.4 mM RbCl₂, 5 mM glucose) and incubated at 37° C. for 2 h. Attached cells were quickly washed three times with buffer (same as Rb⁺ loading buffer, but containing 5.4 mM KCl instead of RbCl) to remove extracellular Rb⁺. Immediately following the wash, 0.2 ml of depolarization buffer (wash buffer plus 15 mM KCl) with or without compounds was added to the cells to activate efflux of potassium ion channels. After incubation for 10 min at room temperature, the supernatant was carefully removed and collected. Cells were lysed by the addition of 0.2 ml of lysis buffer (depolarization buffer plus 0.1% Triton X-100) and the cell lysates were also collected. If collected samples were not immediately analyzed for Rb⁺ contents by atomic absorption spectroscopy (see below), they were stored at 4° C. without any negative effects on subsequent Rb⁺ analysis.

The concentration of Rb⁺ in the supernatants (Rb⁺_{Sup}) and cell lysates (Rb⁺_{Lys}) was quantified using an ICR8000 flame atomic absorption spectrometer (Aurora Biomed Inc., Vancouver, B.C.) under conditions defined by the manufacturer. One 0.05 ml samples were processed automatically from microtiter plates by dilution with an equal volume of Rb⁺ sample analysis buffer and injection into an air-acetylene flame. The amount of Rb⁺ in the sample was measured by absorption at 780 nm using a hollow cathode lamp as light source and a PMT detector. A calibration curve covering the range 0-5 mg/L Rb⁺ in sample analysis buffer was generated with each set of plates. The percent Rb⁺ efflux (F) was defined by

F=[Rb⁺_{Sup}/(Rb⁺_{Sup}+Rb⁺_{Lys})]×100%.

The effect (E) of a compound was defined by:

E=[(F_{c}−F_{b})/(Fₛ−F_{b})]×100%

where the F_{c} is the efflux in the presence of compound in depolarization buffer, F_{b} is the efflux in basal buffer, and Fₛ is the efflux in depolarization buffer, and F_{c} is the efflux in the presence of compound in depolarization buffer. The effect (E) and compound concentration relationship was plotted to calculate an EC₅₀ value, a compound's concentration for 50% of maximal Rb⁺ efflux. The results are shown below. Legend: A: EC50<50 nM; B: EC50=50 nM-200 nM; C: EC50=200 nM-1.0 μM; D: EC50=1.0 μM-10 μM; E: EC50>10 μM

TABLE 1
ACTICVITIES OF EXEMPLARY COMPOUNDS

| COMPOUND | EC50 (nM) |
|---|---|
| 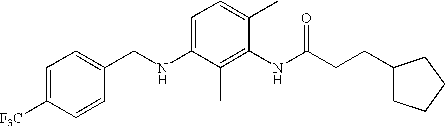 | A |
| 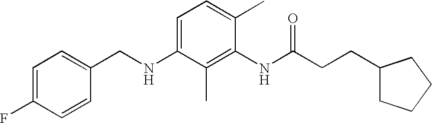 | A |
| 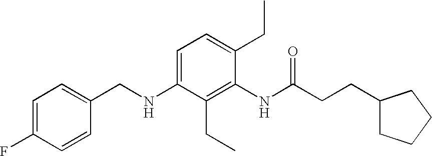 | A |
| 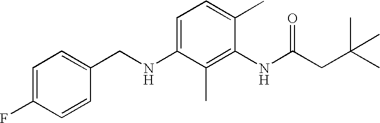 | B |
| 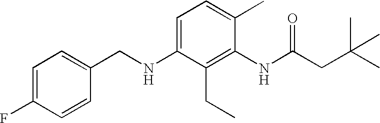 | B |
| 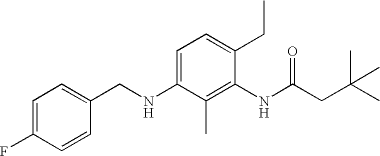 | B |
| 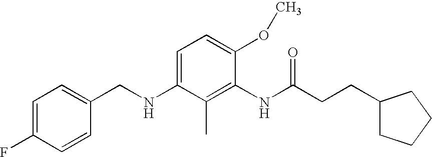 | B |
| 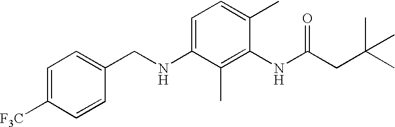 | C |
| 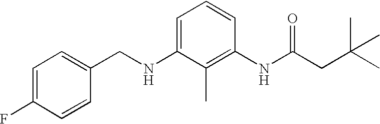 | B |
| 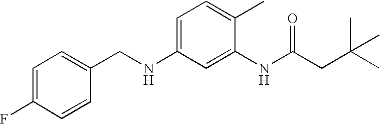 | C |
| 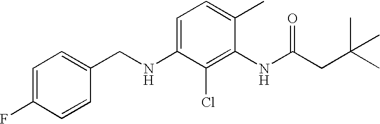 | B |
| 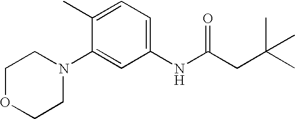 | D |
| 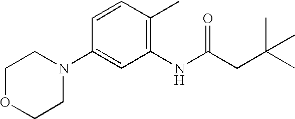 | E |
| 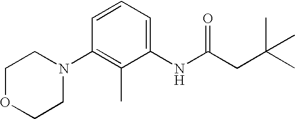 | E |
| 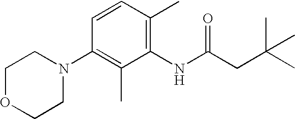 | D |
| 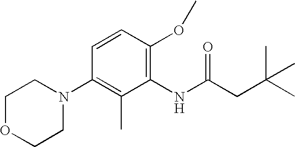 | D |
| 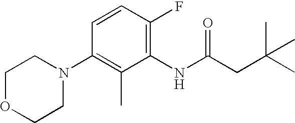 | D |
| 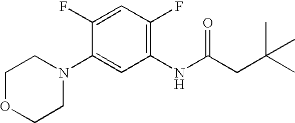 | D |
| 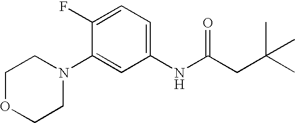 | E |
| 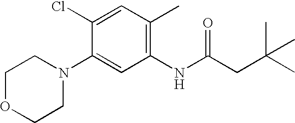 | D |
| 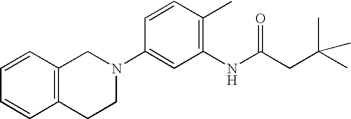 | C |

What is claimed is:

1. A compound of formula I 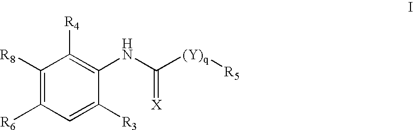
where R₈ is one of Groups A-C below 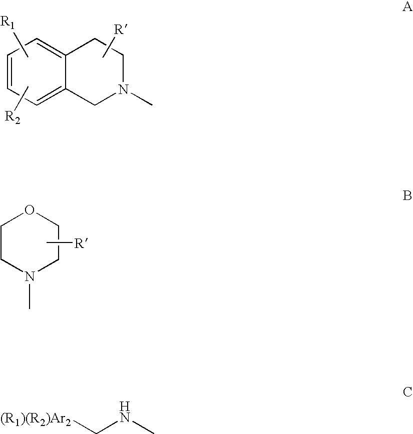
where R₁ and R₂, are, independently, H, CN, halogen, CH₂CN, OH, NO₂, CH₂F, CHF₂, CF₃, CF₂CF₃, C₁-C₆ alkyl, C(═O)C₁-C₆ alkyl, NH—C₁-C₆ alkyl, NHC(═O)C₁-C₆ alkyl, C(═O)N(CH₃)₂, C(═O)N(Et)₂, C(═O)NH—C₁-C₆ alkyl, C(═O)OC₁-C₆ alkyl, OC(═O)C₁-C₆alkyl, OC₁-C₆alkyl, SC₁-C₆alkyl, C₃-C₆ cycloalkyl, (CH₂)ₘC₃-C₆ cycloalkyl, C₃-C₆ cycloalkenyl, (CH₂)ₘC₃-C₆ cycloalkenyl, C₂-C₆ alkenyl, C₂-C₆ alkynyl, phenyl, pyridyl, pyrrolyl, (CH₂)ₘimidazolyl, (CH₂)ₘpyrazyl, (CH₂)ₘoxazolyl, (CH₂)ₘisoxazolyl, (CH₂)ₘthiazolyl, (CH₂)ₘisothiazolyl, (CH₂)ₘphenyl, (CH₂)ₘpyrrolyl, (CH₂)ₘpyridyl, or (CH₂)ₘpyrimidyl, which cycloalkyl and said cycloalkenyl groups optionally contain one or two heteroatoms selected independently from O, N, and S, and which alkyl, cycloalkyl, cycloalkenyl, alkenyl, alkynyl, imidazolyl, pyrazyl, oxazolyl, isoxazolyl, thiazolyl, isothiazolyl, phenyl, pyrrolyl, pyridyl, or pyrimidyl groups are optionally substituted with one or two groups selected, independently, from OH, halogen, cyano, methyl, ethyl, or trifluoromethyl, where m is zero, 1, or 2; or R₁ and R₂, together with the ring carbon atoms to which they are attached, form a 5- or 6-member fused ring, which ring may be saturated, unsaturated, or aromatic, which optionally contains one or two heteroatoms selected independently from O, N, and S, and which is optionally substituted with halogen, CF₃, or C₁-C₃ alkyl; R′ is H, halogen, CF₃, or C₁-C₃ alkyl; R₃, R₄, and R₆ are, independently, H, CN, halogen, CF₃, OCF₃, OC₁-C₃ alkyl, or C₁₋C₆ alkyl, all said C₁-C₃ alkyl groups and said C₁₋C₆ alkyl groups optionally substituted with one or two groups selected, independently, from OH, halogen, C₁-C₃ alkyl, OC₁-C₃ alkyl, or trifluoromethyl; X═O or S; Y is O or S; q=1 or 0; R₅ is C₁-C₆ alkyl, (CHR₇)_{w}C₃-C₆ cycloalkyl, (CHR₇)_{w}CH₂C₃-C₆ cycloalkyl, CH₂(CHR₇)_{w}C₃-C₆ cycloalkyl, CR₇═CH—C₃-C₆ cycloalkyl, CH═CR₇—C₃-C₆ cycloalkyl, (CHR₇)_{w}C₅-C₆ cycloalkenyl, CH₂(CHR₇)_{w}C₅-C₆ cycloalkenyl, C₂-C₆ alkenyl, C₂-C₆ alkynyl, Ar₁, (CHR₇)_{w}Ar₁, CH₂(CHR₇)_{w}Ar₁, or (CHR₇)_{w}CH₂Ar₁, where w=0-3, Ar₁ is a 5- to 10-member mono- or bicyclic aromatic group, optionally containing 1-4 ring heteroatoms selected independently from N, O, and S; R₇ is C₁-C₃ alkyl or hydrogen; Ar₂ is a 5- to 10-member mono- or bicyclic aromatic group, optionally containing 1-4 heteroatoms selected independently from N, O, and S, where all alkyl, cycloalkyl, alkenyl, cycloalkenyl, heterocycloalkyl, heterocycloalkenyl, alkynyl, aryl, and heteroaryl groups in R₁, R₂, R₃, R₄, R₅, R₆, R₇, Ar₁, and Ar₂ are optionally substituted with one or two substituents selected independently from C₁-C₃ alkyl, halogen, OH, OMe, CN, CH₂F, and trifluoromethyl; where, additionally, all cycloalkyl and heterocycloalkyl groups are optionally substituted with either an exocyclic carbon-carbon double bond or a carbonyl group; and where, additionally, the alkenyl and alkynyl groups are also optionally substituted with phenyl or C₃-C₆ cycloalkyl; or a pharmaceutically acceptable salt thereof.

2. The compound of claim 1, where NH—C(═X)—(Y)_{q}—R₅ is NHC(═S)R₅, NHC(═O)R₅, or NHC(═O)OR₅ and where R₁ and R₂, are, independently, phenyl, H, halogen, CF₃, C₁-C₃ alkyl, C₃-C₆ cycloalkyl, (CH₂)ₘC₃-C₆ cycloalkyl, OC₁-C₃ alkyl, or C(═O)C₁-C₆ alkyl.

3. The compound of claim 2, which is a compound of formula IA.

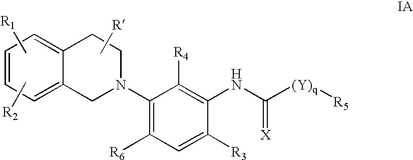

4. The compound of claim 2, which is a compound of formula IB.

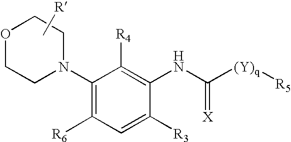

5. The compound of claim 2, which is a compound of formula IC

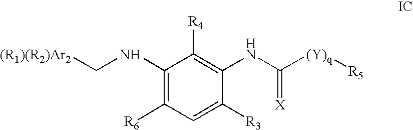

6. The compound of claim 3 which is a compound of formula IA-1

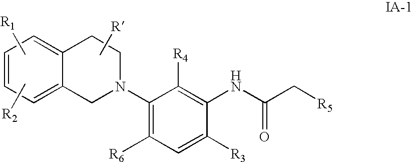

7. The compound of claim 3 which is a compound of formula IA-2

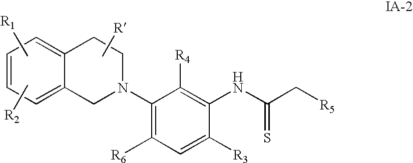

8. The compound of claim 3 which is chosen from compounds IA-3, IA-4, and IA-5

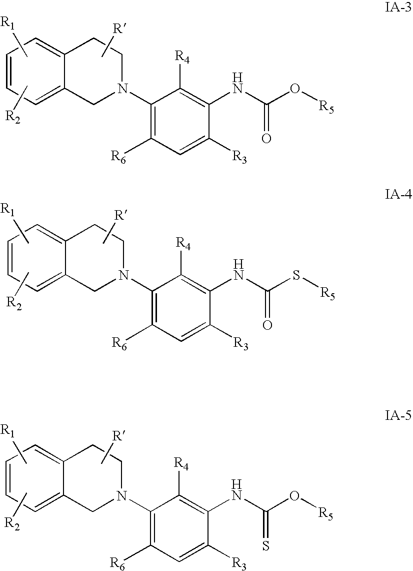

9. The compound of claim 6, where R′ is H, halogen, or C₁-C₃ alkyl, R₂ is H or halogen, R₃ and R₄ are H, halogen, OC₁-C₃ alkyl, ethyl, or methyl, and R₅ is C₅-C₆ alkyl or 2-cyclopentyl ethyl.

10. The compound of claim 9, where R₁ is H, CN, halogen, CH₂CN, OH, OC₁-C₆ alkyl, CH₂F, CHF₂, CF₃, CF₂CF₃, C₁-C₆ alkyl, or C(═O)C₁-C₆ alkyl, and R′ is H.

11. The compound of claim 1, where R₃ and R₄ are both methyl, and (Y)_{q}—R₅ is C₃-C₆ alkyl, O—C₁-C₆ alkyl, (CHR₇)_{w}C₃-C₆ cycloalkyl, (CHR₇)_{w}CH₂C₃-C₆ cycloalkyl, CH₂(CHR₇)_{w}C₃-C₆ cycloalkyl, O—(CHR₇)_{w}C₃-C₆ cycloalkyl, O—(CHR₇)_{w}CH₂C₃-C₆ cycloalkyl, or O—CH₂(CHR₇)_{w}C₃-C₆ cycloalkyl, and where, in substituents A-C, R′ is H.

12. A compound which is one of the following:
N-(5-(3,4-dihydroisoquinolin-2(1H)-yl)-2-methylphenyl)-3,3-dimethyl butanamide
N-(2-fluoro-5-morpholinophenyl)-3,3-dimethylbutanamide
3,3-dimethyl-N-(2-methyl-5-morpholinophenyl)butanamide
3,3-dimethyl-N-(2-methyl-3-morpholinophenyl)butanamide
N-(2,6-dimethyl-3-morpholinophenyl)-3,3-dimethylbutanamide
3-cyclopentyl-N-(3-(4-fluorobenzylamino)-2,6-dimethylphenyl)propanamide
3-cyclopentyl-N-(2,6-dimethyl-3-(4-(trifluoromethyl)benzylamino)phenyl)propanamide
N-[3-(3-cyclopentylpropanamido)-2,4-dimethylphenyl]-3,4-difluorobenzamide
N-(6-ethyl-3-(4-fluorobenzylamino)-2-methylphenyl)-3,3-dimethylbutanamide
N-(2-ethyl-3-(4-fluorobenzylamino)-6-methylphenyl)-3,3-dimethylbutanamide
N-(3-(4-fluorobenzylamino)-2,6-dimethylphenyl)-3,3-dimethylbutanamide
N-(2,6-dimethyl-3-(4-(trifluoromethyl)benzylamino)phenyl)-3,3-dimethyl butanamide
3-cyclopentyl-N-(2,6-diethyl-3-(4-fluorobenzylamino)phenyl)propanamide
2-cyclopentyl-N-(3-(4-fluorobenzylamino)-6-methoxy-2-methylphenyl)acetamide
N-(6-chloro-3-(4-fluorobenzylamino)-2-methylphenyl)-3,3-dimethyl butanamide
N-(4-chloro-3-(4-fluorobenzylamino)-2-methylphenyl)-3,3-dimethyl butanamide
N-(2-chloro-3-(4-fluorobenzylamino)-6-methylphenyl)-3,3-dimethyl butanamide
N-(4-chloro-5-(4-fluorobenzylamino)-2-methylphenyl)-3,3-dimethyl butanamide
N-(5-(4-fluorobenzylamino)-2-methylphenyl)-3,3-dimethylbutanamide
N-(3-(4-fluorobenzylamino)-2-methylphenyl)-3,3-dimethylbutanamide
N-(2,4-difluoro-5-(4-fluorobenzylamino)phenyl)-3,3-dimethylbutanamide and
N-(4-fluoro-3-(4-fluorobenzylamino)phenyl)-3,3-dimethylbutanamide.

13. A composition comprising a pharmaceutically acceptable carrier and a pharmaceutically effective amount of a compound of formula I.

14. A method of treating or preventing a disease or disorder that is ameliorated by a potassium channel opener, comprising administering to a patient in need thereof an effective amount of a compound of formula I.

15. The method of claim 14, where the compound of formula I is a compound of formula IA.

16. A method of treating or preventing in a human or animal a disease or disorder that is ameliorated by a modulation of potassium channels, comprising administering to a patient in need thereof an effective amount of a compound chosen from the following:
N-(5-(3,4-dihydroisoquinolin-2(1H)-yl)-2-methylphenyl)-3,3-dimethyl butanamide
N-(2-fluoro-5-morpholinophenyl)-3,3-dimethylbutanamide
3,3-Dimethyl-N-(2-methyl-5-morpholinophenyl)butanamide
3,3-Dimethyl-N-(2-methyl-3-morpholinophenyl)butanamide
N-(2,6-dimethyl-3-morpholinophenyl)-3,3-dimethylbutanamide
3-Cyclopentyl-N-(3-(4-fluorobenzylamino)-2,6-dimethylphenyl)propanamide
3-cyclopentyl-N-(2,6-dimethyl-3-(4-(trifluoromethyl)benzylamino)phenyl)propanamide
N-[3-(3-cyclopentylpropanamido)-2,4-dimethylphenyl]-3,4-difluorobenzamide
N-(6-ethyl-3-(4-fluorobenzylamino)-2-methylphenyl)-3,3-dimethylbutanamide
N-(2-ethyl-3-(4-fluorobenzylamino)-6-methylphenyl)-3,3-dimethylbutanamide
N-(3-(4-fluorobenzylamino)-2,6-dimethylphenyl)-3,3-dimethylbutanamide
N-(2,6-dimethyl-3-(4-(trifluoromethyl)benzylamino)phenyl)-3,3-dimethyl butanamide
3-Cyclopentyl-N-(2,6-diethyl-3-(4-fluorobenzylamino)phenyl)propanamide
2-Cyclopentyl-N-(3-(4-fluorobenzylamino)-6-methoxy-2-methylphenyl)acetamide
N-(6-chloro-3-(4-fluorobenzylamino)-2-methylphenyl)-3,3-dimethyl butanamide
N-(4-chloro-3-(4-fluorobenzylamino)-2-methylphenyl)-3,3-dimethyl butanamide
N-(2-chloro-3-(4-fluorobenzylamino)-6-methylphenyl)-3,3-dimethyl butanamide
N-(4-chloro-5-(4-fluorobenzylamino)-2-methylphenyl)-3,3-dimethyl butanamide
N-(5-(4-fluorobenzylamino)-2-methylphenyl)-3,3-dimethylbutanamide
N-(3-(4-fluorobenzylamino)-2-methylphenyl)-3,3-dimethylbutanamide
N-(2,4-difluoro-5-(4-fluorobenzylamino)phenyl)-3,3-dimethylbutanamide and
N-(4-fluoro-3-(4-fluorobenzylamino)phenyl)-3,3-dimethylbutanamide.
